(19) United States
(12) Patent Application Publication
(10) Pub. No.: US 2010/0113460 A1
(43) Pub. Date: May 6, 2010

(54) Organic Compounds

(75) Inventors: Boris Schilling, Knonau (CH); Wolf D. Woggon, Binningen (CH); Antoinette Chougnet, Binningen (CH); Thierry Granier, Duebendorf (CH); Georg Frater, Winterthur (CH); Andreas Hanhart, Uster (CH)

Correspondence Address: PARFOMAK, ANDREW N.;NORRIS MCLAUGHLIN & MARCUS PA, 875 THIRD AVE, 8TH FLOOR, NEW YORK, NY, 10022, US

(73) Assignee: Givaudan SA, Vernier (CH)

(21) Appl. No.: 12/532,758

(22) PCT Filed: Mar. 20, 2008
(86) PCT No.: PCT/CH2008/000129

§ 371 Date: Sep. 23, 2009

(30) Foreign Application Priority Data
Mar. 28, 2007 (GB) 0705931.4

(51) Int. Cl. A61K 31/4965; A24B 15/00; A61K 31/395; A61K 31/498; A61K 31/517; A61K 31/435; A61K 31/423; A61K 31/4196; A61K 31/4184; A61K 31/4164; A61K 31/4015; A61K 31/381; A61K 31/353; A61K 31/366; A61K 31/357; A61K 31/215; A61K 31/12; A61K 31/122; C07D 225/02; C07D 487/04; C07D 239/70; C07D 221/04; C07D 263/52; C07D 233/58; C07D 207/24; C07D 309/30; C07D 317/26; C07D 307/28; C07C 255/50; C07C 49/21; C07C 49/255; C07C 49/213; C07C 49/215; C07C 49/623; C07C 49/395

(52) U.S. Cl. 514/249; 131/352; 514/183; 514/252.1; 514/258.1; 514/299; 514/375; 514/383; 514/393; 514/396; 514/424; 514/438; 514/457; 514/460; 514/462; 514/467; 514/473; 514/530; 514/675; 514/678; 514/682; 514/690; 514/691; 540/451; 540/476; 544/253; 546/112; 548/217; 548/335.1; 548/534; 548/543; 549/265; 549/273; 549/454; 549/498; 558/414; 568/303; 568/308; 568/328; 568/374; 568/379

(57) ABSTRACT

Disclosed are compounds having the ability to inhibit cytochrome P450 2A6, 2A13, and/or 2B6 and tobacco products comprising them. Also disclosed are pharmaceutical compositions comprising them.

The present invention refers to compounds useful in methods of inhibiting cytochrome P450 2A6, 2A13 and/or 2B6, and to products comprising them.

It is known from the art that inhibition of cytochrome P450 enzyme CYP2A6/2A13 and CYP2B6 reduces nicotine metabolism in a subject in which nicotine is present, thereby increasing blood levels of nicotine and predisposing the subject to ingest lower amounts of nicotine. It is also known, that inhibition of cytochrome P450 enzymes CYP2A and CYP2B6 are useful for decreasing metabolism of other products, including, for example, promutagens that are activated by CYP2A to mutagens. For example, inhibition of CYP2A is useful for preventing mutagenic activation of the carcinogenic, tobacco-specific promutagen 4-(methylnitrosaminio)-1-(3-pyridyl)-1-butanone (NNK), thereby decreasing the risk of developing cancer. NNK is formed during the processing and curing of tobacco plants by nitrosation, and it is also believed that nicotine could be converted endogenously to NNK. It is present in tobacco and in tobacco smoke, both mainstream and in sidestream smoke. NNK is a procarcinogen which is metabolically activated by alpha-hydroxylation catalysed by cytochrome P450 activity and the resulting reactive electrophilic metabolites ultimately alkylate DNA.

CYP2A13 is one of three members of the human CYP2A family. The other two are CYP2A6 and CYP2A7. Whereas CYP2A6 seems to be a major human liver metabolic enzyme, which also hydroxylates coumarin and metabolises nicotine to cotinine, for CYP2A7 a catalytic activity is presently unknown and it is believed to be a pseudogene. CYP2A6 is also detected in the human respiratory tract, but CYP2A13 is the dominantly expressed isoform in the human nose and the respiratory tract, however, other P450 enzymes also contribute to metabolism. In particular CYP2A6 and CYP2B6 are prone to metabolize small molecular weight substrates. CYP2B6 also has been identified as being the second important catalyst besides CYP2A13 which is metabolically activating tobacco-specific nitrosamines, such as NNK.

Surprisingly there has been found a new class of chemical compounds capable of inhibiting the enzyme activity of CYP2A, such as, CYP2A6 and CYP2A13, and CYP2B6 thus making them very suitable in combination with tobacco products for the reduction or inhibition of the metabolism of NNK in the respiratory tract when inhaled together with tobacco smoke.

Accordingly, the present invention refers in one of its aspects to a tobacco product, such as cigarettes, chewing tobacco, snuff tobacco, pipe tobacco and cigars, comprising a compound of formula (I)

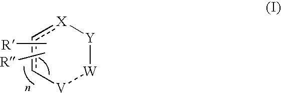

wherein
n is 0 or an integer from 1 to 12, e.g. 3, 4, 5, 6, 8 or 9;
the dashed lines representing independently a bond or no bond;
R′ is H, C₁-C₁₀ alkyl, C₂-C₁₀ alkenyl, —CH₂—C(O)—(C₁-C₁₀)alkyl, or —(CH₂)ₖ—COO—(C₁-C₁₀)alkyl, wherein k is 0 or 1; and
R″ is H, C₁-C₁₀ alkyl; or
R′ and R″ together represent a bivalent group —(CH₂)ₐ— wherein “a” is 1-5 (e.g. 2, 3 or 4), forming together with the carbon atom(s) to which they are attached cycloalkyl (e.g. cyclopropan, cyclobutan, cyclohexan, cyclopentan) optionally substituted with C₁-C₃ alkyl, e.g methyl and ethyl, or C₁-C₃ alkoxy, e.g. ethoxy;
I) —X—Y— represents a bivalent group selected from

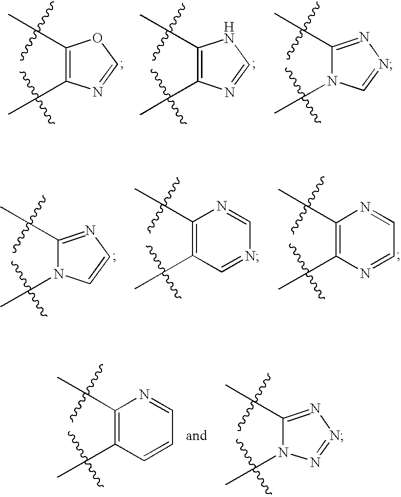

II) Y is carbonyl and
   X is O, NH, CHR², CR² wherein R² is H, C₁-C₁₀ alkyl, C₂-C₁₀ alkenyl, C₂-C₁₀ alkynyl, or —COO—R¹¹ wherein R¹¹ is C₁-C₁₀ alkyl or C₂-C₁₀ alkenyl, or X is NR³, wherein R³ is C₁-C₁₀ alkyl or C₂-C₁₀ alkenyl;
      or
III) Y is —CR¹═, wherein R¹ is H, C₁-C₁₀ alkyl (linear or branched), C₂-C₁₀ alkenyl (linear or branched), or cycloalkylalkyl (e.g. cyclopentylmethyl); and
   X is O, N, or NR⁵, wherein R⁵ is H, C₁-C₁₀ hydroxyalkyl, C₁-C₁₀ cyanoalkyl, C₁-C₁₀ alkyl (linear or branched), C₂-C₁₀ alkenyl (linear or branched), C₂-C₁₀ alkynyl, —(CH₂)ₘ—COO—R¹², wherein m is 1, 2, 3, 4, or 5 and R¹² is H, or C₁-C₁₀ alkyl;
      and
i) if the dashed line V [symbol] W represents a bond then
   V is selected from O, N, —CH₂—, —CR⁴═ wherein R⁴ is H, or C₁-C₃ alkyl, —CR⁶R⁷— wherein R⁶ is H, or C₁-C₆ alkyl, and R⁷ is H, or R⁷ and R′ together represent a bivalent group selected from —O— and —CH₂— forming a 3-membered ring; and
   W represents a direct bond from Y to V, or is —CH₂—, —CHR″— or —CH═;
ii) if the dashed line V [symbol] W is no bond;
   W is C₁-C₃ alkyl, C₂-C₇ alkenyl (e.g. 3-methyl but-2-en-1-yl), cycloalkylvinyl comprising from 5 to 7 carbon atoms (e.g. cyclopropylethenyl), arylvinyl comprising 5 to 7 carbon atoms (e.g. phenylethylene), phenyl, C₁-C₃ alkoxy (e.g. methoxy or ethoxy), or C₂-C₃ alkenyloxy (e.g. —O—CH₂—CH═CH₂); and
   A) V is CR⁸R⁹R¹⁰ wherein R⁸, R⁹, R¹⁰ are hydrogen, R⁸ and R⁹ are methyl and R¹⁰ is hydrogen or methyl; or R⁸ and R⁹ representing independently H, or C₁-C₆ alkoxy (e.g. ethoxy) and R¹⁰ is C₁-C₆ alkoxy (e.g. ethoxy);
   B) V is a 3-6 membered monocyclic or 6-10 membered bicyclic hydrocarbon ring (e.g. cyclopropyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N (e.g. furanyl, thienyl, tetrahydrofuranyl);
   C) V is a 3-6 membered monocyclic or 6-10 membered bicyclic hydrocarbon ring (e.g. cyclopropyl, cyclopentyl, cyclopentadienyl, cyclopentenyl, cyclohexenyl, cyclohexyl, phenyl, naphtyl) wherein up to two, i.e. 0, 1 or 2, atom(s) are replaced by a hetero atom selected from S, O, and N, and the ring is substituted with one or two groups selected from CN, halogen (e.g. F, Cl, Br), C₁-C₃ alkoxy (e.g. methoxy, ethoxy), C₁-C₃ alkyl and —COOR, wherein R is hydrogen, methyl, ethyl, propyl or isopropyl;
   D) V is a bivalent residue —CH₂—CH₂— forming together with the carbon atom of X which is in alpha-position to the carbonyl group (Y) a cyclobutan or cyclopentan ring; or
   E) V is —C(O)R¹³ wherein R¹³ is C₁-C₃ alkyl, or C₁-C₃ alkoxy.

Preferably the compounds of formula (I) comprise one, two or three ring(s).

Non-limiting example compounds may be selected from the group of compounds of formula (I) wherein R′ and R″ are hydrogen, n is an integer from 3 to 11, e.g. 4, 6, 7, 8 or 9, the dashed line V [symbol] W represents a bond wherein W and V are —CH₂—;

Y is carbonyl and X is NH; or
—X—Y— represents a bivalent group selected from

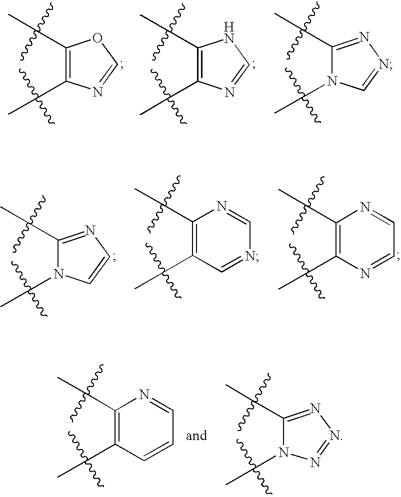

Specific examples of these include
4,5,6,7,8,9,10,11,12,13-decahydrocyclododeca[d]oxazole (Compound ID 15);
5,6,7,8,9,10,11,12,13,14-decahydrocyclododeca[d]pyrimidine (Compound ID 1)
5,6,7,8,9,10,11,12,13,14,15,16-dodecahydro-4H-cyclopentadeca[d]oxazole;
4,5,6,7,8,9-hexahydrocycloocta[d]oxazole (Compound ID 60);
5,6,7,8,9,10,11,12-octahydrocyclodeca[d]pyrimidine (Compound ID 26);
5,6,7,8,9,10,11,12-octahydro-4H-cycloundeca[d]oxazole (Compound ID 14);
1,4,5,6,7,8,9,10,11,12-decahydrocycloundeca[d]imidazole (Compound ID 35);
4,5,6,7,8,9,10,11,12,13-decahydro-1H-cyclododeca[d]imidazole (Compound ID 59);
6,7,8,9,10,11,12,13,14,15-decahydro-5H-[1,2,4]triazolo[4,3-a][1]azacyclotridecine (Compound ID 43);
5,6,7,8,9,10,11,12,13,14-decahydrocyclododeca[b]pyrazine (Compound ID 42);
5,6,7,8,9,10,11,12,13,14-decahydrocyclododeca[b]pyridine (Compound ID 24);
5,6,7,8,9,10,11,12,13,14-decahydroimidazo[1,2-a][1]azacyclododecine (Compound ID 53);
6,7,8,9,10,11,12,13,14,15-decahydro-5H-imidazo[1,2-a][1]azacyclotridecine (Compound ID 64);
azacyclotridecan-2-one (Compound ID 36); and
azacyclododecan-2-one (Compound ID 65).

Alternatively, the compounds of formula (I) are those wherein n is 0 or 1;

the dashed line V [symbol] W represents a bond;
W represents a direct bond from Y to V, or is —CH₂—, —CHR″— or —CH═;
V is selected from O, N, —CH₂—, —CR⁴═ wherein R⁴ is H, or C₁-C₃ alkyl, —CR⁶R⁷— wherein R⁶ is H, or C₁-C₆ alkyl, and R⁷ is H, or R⁷ and R′ together represent a bivalent group selected from —O— and —CH₂— forming a 3-membered ring;
R′ is H, C₁-C₁₀ alkyl, C₂-C₁₀ alkenyl, —CH₂—C(O)—(C₁-C₁₀)alkyl, or —(CH₂)ₖ—COO—(C₁-C₁₀)alkyl, wherein k is 0 or 1; and
R″ is H, C₁-C₁₀ alkyl; or
R′ and R″ together represent a bivalent group —(CH₂)ₐ— wherein a is 1-5 (e.g. 2, 3 or 4), forming together with the carbon atom(s) to which they are attached a cycloalkyl (e.g. cyclopropan, cyclobutan, cyclohexan, cyclopentan) optionally substituted with C₁-C₃ alkyl, e.g. methyl and ethyl, or C₁-C₃ alkoxy, e.g. ethoxy; and
I) Y is carbonyl and
   X is O, NH, CHR², CR² wherein R² is H, C₁-C₁₀ alkyl, C₂-C₁₀ alkenyl, C₂-C₁₀ alkynyl, or —COO—R¹¹ wherein R¹¹ is C₁-C₁₀ alkyl or C₂-C₁₀ alkenyl, or X is NR³, wherein R³ is C₁-C₁₀ alkyl or C₂-C₁₀ alkenyl; or
II) Y is —CR¹═, wherein R¹ is H, C₁-C₁₀ alkyl (linear or branched), C₂-C₁₀ alkenyl (linear or branched), or cycloalkylalkyl (e.g. cyclopentylmethyl);
   X is O, N, or NR⁵, wherein R⁵ is H, C₁-C₁₀ hydroxyalkyl, C₁-C₁₀ cyanoalkyl, C₁-C₁₀ alkyl (linear or branched), C₂-C₁₀ alkenyl (linear or branched), C₂-C₁₀ alkynyl, —(CH₂)ₘ—COO—R¹², wherein m is 1, 2, 3, 4, or 5 and R¹² is H, or C₁-C₁₀ alkyl.

Non-limiting example compounds may be selected from the group of compounds of formula (I) wherein Y is carbonyl and R′ is H, C₁-C₁₀ alkyl, e.g. methyl, n-butyl, n-pentyl, n-hexyl, or C₂-C₁₀ alkenyl, i.e. C₂, C₃, C₄, C₅, C₆, C₇, C₈, C₉ or C₁₀alkenyl, e.g. pent-2-en-1-yl, pen-3-en-1-yl, hex-3-en-1-yl; and R″ is H, C₁-C₁₀ alkyl; or R′ and R″ together represent a bivalent group —(CH₂)ₐ— wherein a is 1-5 (e.g. 2, 3 or 4), forming together with the carbon atom(s) to which they are attached cycloalkyl (e.g. cyclopropan, cyclohexan) optionally substituted with C₁-C₃ alkyl, e.g methyl and ethyl, or C₁-C₃ alkoxy, e.g. ethoxy; and

i) X is oxygen; the dashed line V [symbol] W represents a bond wherein W represents a direct bond from Y to V and V is —CH₂—; n is 1 or 2, or
ii) X is CHR² wherein R² is hydrogen; the dashed line V [symbol] W represents a bond wherein W represents a direct bond from Y to V and V is oxygen; n is 1 or 2; or
iii) X is oxygen; the dashed line V [symbol] W represents a bond; W and V are —CH₂—; n is 0 or 1; or
iv) X is CHR² wherein R² is hydrogen; the dashed line V [symbol] W represents a bond wherein W represents a direct bond from Y to V and V is oxygen; n is 0 or 1.

Compounds of formula (I) wherein Y is carbonyl and either X or V is oxygen, i.e. lactone derivatives as defined herein above may be selected from the group consisting of 5-hexyldihydrofuran-2(3H)-one (Compound ID 47); 3-pentyltetrahydro-2H-pyran-2-one; 4-methyl-5-pentyldihydrofuran-2(3H)-one (Compound ID 12); (Z)-3-(pent-3-enyl)tetrahydro-2H-pyran-2-one (Compound ID 58); octahydrocoumarin (Compound ID 45); 5-hexyl-5-methyldihydrofuran-2(3H)-one (Compound ID 31); 5-butyldihydrofuran-2(3H)-one (Compound ID 63); (Z)-6-(pent-2-enyl)tetrahydro-2H-pyran-2-one (Compound ID 22); 8-ethyl-1-oxaspiro[4.5]decan-2-one (Compound ID 29); 4-methyl-5-butyldihydrofuran-2(3H)-one (Compound ID 11); 8-methyl-1-oxaspiro[4.5]decan-2-one (Compound ID 69); and (E/Z)-5-(hex-3-enyl)-5-methyldihydrofuran-2(3H)-one (Compound ID 62).

Further non-limiting example compounds may be selected from the group of compounds of formula (I) wherein Y is carbonyl, X is CHR² or CR² wherein R² is C₁-C₁₀ alkyl, e.g n-butyl, n-pentyl or n-hexyl, or C₂-C₁₀ alkenyl, e.g. pent-2-en-1-yl, oct-2-en-1-yl, the dashed line V [symbol] W represents a bond, W and V are —CH₂— n is 0, R″ is H, and R′ is H, C₁-C₁₀ alkyl, e.g. methyl, n-butyl, n-pentyl, n-hexyl, or —(CH₂)ₖ—COO—(C₁-C₁₀)alkyl, wherein k is 0 or 1, e.g. methylacetate.

Specific examples of these include
2-hexyl-3-methylcyclopent-2-enone (Compound ID 3),
2-pentyl-3-methylcyclopent-2-enone (Compound ID 7),
(Z)-3-methyl-2-(pent-2-enyl)cyclopent-2-enone (Compound ID 10),
(3-methyl-2-(pent-2-enyl)cyclopentanone (Compound ID17),
(E)-2-(oct-2-enyl)cyclopentanone (Compound ID 18),
(Z)-methyl 2-(3-oxo-2-(pent-2-enyl)cyclopentyl)acetate (Compound ID 25),
2-hexylcyclopentanone (Compound ID 27),
2-hexylcyclopent-2-enone (Compound ID 30),
3-methyl-2-pentylcyclopentanone (Compound ID 32),
3-methyl-2-butylcyclopentanone (Compound ID 34),
methyl 2-(3-oxo-2-pentylcyclopentyl)acetate (Compound ID 37), and
2-pentylcyclopent-2-enone (Compound ID 51).

Further non-limiting example compounds may be selected from N-substituted imidazoles, i.e. compounds of formula (I) wherein R′ and R″ are hydrogen, n is 1, the dashed line V [symbol] W represents a bond wherein W represents a direct bond from Y to V and V is N, Y is —CR¹═ wherein R¹ is hydrogen, and X is NR⁵, wherein R⁵ is C₁-C₁₀ hydroxyalkyl, C₁-C₁₀ cyanoalkyl, e.g. cyanobutyl, C₁-C₁₀ alkyl (linear or branched), such as n-pentyl, n-hexyl, 3-methyl-but-1-yl, C₂-C₁₀ alkenyl (linear or branched), such as pent-2-en-1-yl, hex-3-en-1yl, 3-methyl-but-2-en-1-yl, hex-5-en-1-yl, 3,7-dimethyl-oct-2,6-dien-1-yl, C₂-C₁₀ alkynyl, —(CH₂)ₘ—COO—R¹², wherein m is 1, 2, 3, 4, or 5 and R¹² is H, or C₁-C₁₀ alkyl, e.g. —(CH₂)₃—COO—C₂H₅.

Specific examples of these compounds include
4-(1H-imidazol-1-yl)butan-1-ol (Compound ID 68)
ethyl 4-(1H-imidazol-1-yl)butanoate (Compound ID 49),
1-(3,7-dimethylocta-2,6-dienyl)-1H-imidazole (Compound ID 44)
1-isopentyl-1H-imidazole (Compound ID 39)
1-pentyl-1H-imidazole (Compound ID 28)
(E)-1-(hex-3-enyl)-1H-imidazole (Compound ID 46)
1-(3-methylbut-2-enyl)-1H-imidazole (Compound ID 40)
1-hexyl-1H-imidazole (Compound ID 16)
5-(1H-imidazol-1-yl)pentanenitrile (Compound ID 55)
1-(hex-5-enyl)-1H-imidazole (Compound ID 41)
(Z)-1-(hex-3-enyl)-1H-imidazole (Compound ID 23), and
(Z)-1-(pent-2-enyl)-1H-imidazole (Compound ID 54).

In another alternative, the compounds of formula (I) are those wherein n is 0 or 1;

the dashed lines represent independently a bond or no bond with the proviso that the dashed line V [symbol] W is no bond;

### R′ is H; and

R″ is H, or C₁-C₄ alkyl; or
R′ and R″ together represent a bivalent group —(CH₂)ₐ— wherein “a” is 1-5 (e.g. 2, 3 or 4), forming together with the carbon atoms to which they are attached a cycloalkyl (e.g. cyclopropan, cyclobutan, cyclohexan, cyclopentan);
Y is carbonyl;
X is CHR² or CR² wherein R² is H, C₁-C₁₀ alkyl, e.g. C₂, C₃, C₄ or C₇ linear or branched alkyl;
W is C₁-C₃ alkyl, C₂-C₇ alkenyl (e.g. 3-methyl but-2-en-1yl), cycloalkylvinyl comprising from 5 to 7 carbon atoms (e.g. cyclopropylethenyl), arylvinyl comprising 5 to 7 carbon atoms (e.g. phenylethylene), phenyl, C₁-C₃ alkoxy (e.g. methoxy or ethoxy), or C₂-C₃ alkenyloxy (e.g. —O—CH₂—CH═CH₂); and
A) V is CR⁹R⁹R¹⁰ wherein R⁸, R⁹, R¹⁰ are hydrogen, R⁸ and R⁹ are methyl and R¹⁰ is hydrogen or methyl; or R⁸ and R⁹ representing independently H, or C₁-C₆ alkoxy (e.g. ethoxy) and R¹⁰ is C₁-C₆ alkoxy (e.g. ethoxy);
B) V is a 3-6 membered monocyclic or 6-10 membered bicyclic hydrocarbon ring (e.g. cyclopropyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N (e.g. furanyl, thienyl, tetrahydrofuranyl);
C) V is a 3-6 membered monocyclic or 6-10 membered bicyclic hydrocarbon ring (e.g. cyclopropyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N, and the ring is substituted with one or two groups selected from CN, halogen (e.g. F, Cl, Br), C₁-C₃ alkoxy (e.g. methoxy, ethoxy), C₁-C₃ alkyl and —COOR, wherein R is hydrogen, methyl, ethyl, propyl or is isopropyl;
D) V is a bivalent residue —CH₂—CH₂— forming together with the carbon atom of X which is in alpha-position to the carbonyl group (Y) a cyclobutan or cyclopentan ring; or
E) V is —C(O)R¹³ wherein R¹³ is C₁-C₃ alkyl, or C₁-C₃ alkoxy.

Non-limiting example compounds may be selected from the group of compounds of formula (I) wherein Y is carbonyl, dashed line V [symbol] W is no bond, W is CH₃ or cyclopropylethenyl, X is CR² or CHR² wherein R² is C₃-C₁₀ alkyl, e.g. C₄, C₅ or C₆ linear alkyl, n is 0 or 1, and

V is cyclopropyl, phenyl, naphthyl, furanyl, thienyl, tetrahydrofuranyl, 2-methyl dioxolan-2-yl, or
phenyl substituted with one or two groups selected from CN, halogen (e.g. F, Cl, Br), C₁-C₃ alkoxy (e.g. methoxy, ethoxy), C₁-C₃ alkyl and —COOR, wherein R is hydrogen, methyl, ethyl, propyl or is isopropyl, or
V is CR⁸R⁹R¹⁰ wherein R⁸ is hydrogen and R⁹ and R¹⁰ representing independently C₁-C₆ alkoxy, such as methoxy or ethoxy.

Specific examples of these include
(E)-3-(cyclopropylmethylene)octan-2-one (Compound ID 2);
(E)-3-(cyclopropylmethylene)heptan-2-one (Compound ID 4);
(E)-3-(cyclopropylmethylene)nonan-2-one (Compound ID 6);
(1E,4E)-1-cyclopropyl-4-(cyclopropylmethylene)dec-1-en-3-one (Compound ID 56);
(E)-3-benzylideneheptan-2-one;
(E)-3-benzylideneoctan-2-one (Compound ID 5);
(1E,4E)-4-benzylidene-1-phenylnon-1-en-3-one;
(E)-3-benzylidenenonan-2-one (Compound ID 20);
3-phenylmethylheptan-2-one;
3-phenylmethyloctan-2-one (Compound ID 33);
(E)-4-(2-acetylhept-1-enyl)-benzonitrile (Compound ID 13);
(E)-3-(naphthalen-2-ylmethylene)octan-2-one (Compound ID 67);
(E)-3-(thiophen-2-ylmethylene)octan-2-one (Compound ID 38);
(E)-3-(furan-2-ylmethylene)octan-2-one;
3-((tetrahydrofuran-2-yl)methypoctan-2-one;
(E)-3-((tetrahydrofuran-3-yl)methylene)heptan-2-one (Compound ID 66);
(E)-3-((tetrahydrofuran-3-yl)methylene)octan-2-one;
3-((tetrahydrofuran-3-yl)methyl)octan-2-one;
(E)-3-(2,2-dimethoxyethylidene)heptan-2-one;
(E)-3-(2,2-dimethoxyethylidene)-octan-2-one (Compound ID 9);
3-(2,2-dimethoxyethyl)octan-2-one;
3-(2-methoxyethyl)octan-2-one;
(E)-3-(2-(2-methyl-1,3-dioxolan-2-ypethylidene)octan-2-one (Compound ID 21);
3-(2-(2-methyl-1,3-dioxolan-2-ypethypoctan-2-one;
3-pentylheptane-2,6-dione;
(E)-3-ethylideneoctan-2-one;
1-(2-methyl-1-pentylcyclopropypethanone;
3-(propan-2-ylidene)octan-2-one;
methyl 1-pentylcyclopentanecarboxylate and
1-(1-pentylcyclopentyl)ethanone.

As used in relation to compounds of formula (I), unless otherwise indicated “alkyl” refers to linear or branched C₁ to C₁₀ alkyl, preferably C₁ to C₆, e.g. methyl, ethyl, i-propyl, n-propyl, n-butyl, sec-butyl, tert-butyl, n-pentyl, sec.pentyl, tert-pentyl, n-hexyl, 3-methyl but-1-yl;

“alkenyl” refers to C₂ to C₁₀ alkenyl, preferably linear or branched C₄ to C₈ alkenyl comprising one, two or more double bonds, e.g. C₅, C₆ or C₇ alkenyl, such as vinyl, propen-1-yl, propen-2-yl, allyl, 3-methyl but-2-en-1-yl, 3,7-dimethyl oct-2,6-dien-1-yl, pent-2-en-1-yl, pent-3-en-1-yl, hex-3-en-1yl, pent-2-en-1-yl, oct-2-en-1yl, hex-5-en-1-yl, hept-6-en-1-yl;
“alkynyl” refers to linear or branched C₂ to C₁₀ alkynyl, preferably linear C₃ to C₆ alkynyl, e.g. pent-2-yn-1-yl, but-2-yn-1-yl;
“alkoxy” refers to C₁ to C₁₀ alkoxy, preferably C₁ to C₇ alkoxy, e.g. methoxy, ethoxy, propoxy.

The inhibitors, i.e. compounds of formula (I), can be added to or mixed with a tobacco product according to methods known to the person skilled in the art. Typically, they can be sprayed or dripped on to processed or dried whole tobacco or can be used in the form of a dip or solution into which the processed or raw tobacco is placed.

Instead of adding or mixing the inhibitor with the tobacco product, the tobacco paper or filter may comprise at least pne compound of formula (I).

The amount required to produce the desired effect may depend on various factors, including the activity and the volatility. Amounts from about 0.1 to 5% by weight of a compound of formula (I) or mixtures thereof, such as about 0.3 to 2% by weight, e.g. about 1% by weight based on the end product may be sufficient to achieve an effect.

Furthermore, it is assumed that, if inhaled in the presence of tobacco smoke (passive smoker) which comprises NNK, the compounds of formula (I) reduce the NNK metabolic activation, because of their properties as inhibitor for CYP2A and CYP2B enzymes.

Accordingly, the present invention refers in a further aspect to a method comprising the step of disseminating a compound of formula (I) as defined hereinabove into a room comprising tobacco smoke. Any means capable of disseminating a volatile substance into the atmosphere may be used. The use in this specification of the term “means” includes any type of air-freshener devices which may include a heater and/or fan and nebulization systems well known to the person skilled in the art.

Due to the fact that the compounds of formula (I) inhibit the enzyme activity of CYP2A, e.g. CYP2A6 and CYP2A13, and CYP2B6 they may also be used for the regulation of nicotine metabolism in an individual, such as a nicotine replacement therapy.

Accordingly, the present invention refers in a further of its aspects to the preparation of a pharmaceutical composition comprising a compound of formula (I) as defined hereinabove.

The compounds of the present invention can be administered for, for example, oral, nasal, topical, parenteral, local or inhalant use. Oral administration includes the administration in form of tablets, capsules, chewing gums, sprays, and lozenge.

The compounds of the invention can be readily prepared by methods known to the person skilled in the art.

The invention is now further described with reference to the following non-limiting examples. These examples are for the purpose of illustration only and it is understood that variations and modifications can be made by one skilled in the art.

### EXAMPLE 1

### 6,7,8,9,10,11,12,13,14,15-decahydro-5H-[1,2,4]triazolo[4,3-a][1]aza-cyclotridecine (Compound ID 43)

At 20° C., a solution of laurinolactam (10 g, 0.0507 mol) in dichloromethane (150 ml) was treated with triethyloxonium tetrafluoroborate (28.9 g, 0.152 mol). The resulting mixture was stirred for 17 h, cooled to 2° C., treated dropwise with triethylamine (71 ml), stirred for 45 min., and poured into a cooled sodium bicarbonate solution (200 ml). The organic phase was washed with water (100 ml) and with aqueous NaCl solution (100 ml). The aqueous phase was extracted with dichloromethane (30 ml). The combined organic phases were dried (MgSO₄) and the solvent evaporated. The residue (10.8 g) was dissolved in ethanol (100 ml) and treated, at 20° C., with formylhydrazine (8.1 g, 90%, 0.122 mol) and 4 Å molecular sieves (1 g). The resulting mixture was stirred at 50° C. for 24 hours, filtered, and the solvent evaporated. The residue was treated with dichloromethane and water and stirred for 20 min. The organic phase was washed with water. The aqueous phase was extracted with dichloromethane and the combined organic phases were dried (MgSO₄) and the solvent evaporated. FC (700 g SiO₂, ethyl acetate/methanol 6:1) of the crude product (8.5 g) gave the desired bicyclic triazole (2.8 g, 25%).

¹H-NMR (400 MHz, CDCl₃): [symbol] 8.05 (s, H—C═N), 3.95 (t, J=7.2, CH₂N), 2.84 (t, J=7.3, CH₂C═N), 1.92-1.83 (m, 4H), 1.45-1.28 (m, 12H), 1.22-1.14 (m, 2H).

¹³C-NMR (100 MHz, CDCl₃): [symbol] 153.95 (s), 143.43 (d), 43.06 (t), 27.62 (t), 25.21 (t), 25.19 (t), 25.13 (t), 25.01 (t, 3 C), 24.94 (t), 23.78 (t), 23.10 (t), 22.80 (t).

MS (EI): 222 (34), 221 (55), 220 (21), 206 (29), 192 (27), 180 (79), 178 (38), 166 (29), 164 (34), 152 (47), 150 (28), 138 (73), 136 (42), 125 (32), 124 (100), 122 (50), 111 (52), 110 (50), 97 (91), 96 (25), 84 (43), 55 (31), 41 (31).

IR: νₘₐₓ 3091, 2929, 2861, 2843, 1515, 1504, 1465, 1444, 1373, 1348, 1214, 1192, 984, 887, 817, 771, 736, 670 cm⁻¹.

UV (MeOH): λ(log ε) 237 (1.0).

### EXAMPLE 2

### 5,6,7,8,9,10,11,12,13,14-decahydroimidazo[1,2-a][1]azacyclododecine (Compound ID 53)

At 20° C., a solution of cycloundecanone (10 g, 0.0594 mol) in formic acid (60 ml) was treated with hydroxylamine-O-sulfonic acid (11.2 g, 0.0891 mol). The resulting mixture was stirred for 5.5 hours at reflux, cooled, poured into ice-cold water (100 ml), treated with conc. sodium hydroxide (60 ml) and extracted twice with ethyl acetate (100 ml). The organic phase was washed with an aqueous sodium bicarbonate solution (100 ml) and twice with an aqueous NaCl solution (100 ml), dried (MgSO₄) and the solvent evaporated giving the crude azacyclododecan-2-one (10.1 g). At 20° C., a solution of crude azacyclododecan-2-one (5 g, 0.027 mol) in dichloromethane (50 ml) was treated with triethyloxonium tetrafluoroborate (13.7 g, 0.072 mol) and the resulting mixture was stirred for 18 h, and poured into a cooled aqueous sodium bicarbonate solution (400 ml). The organic phase was washed with water (100 ml) and the combined aqueous phases were extracted with ethyl acetate (50 ml). The combined organic phases were dried (MgSO₄) and the solvent evaporated. The residue (4.7 g) was dissolved in methanol (50 ml) and treated, at 20° C., with aminoacetaldehyde dimethyl acetal (9.4 g, 0.089 mol) and 4 Å molecular sieves (1 g). The resulting mixture was stirred at 60° C. for 48 h, filtered, and the solvent evaporated giving 6.77 g of crude N-(azacyclododecan-2-ylidene)-2,2-dimethoxyethanamine.

A solution of crude N-(azacyclododecan-2-ylidene)-2,2-dimethoxyethanamine (3.0 g) in toluene (30 ml) was treated with p-toluenesulfonic acid monohydrate (3.6 g, 19 mmol) and 4 Å molecular sieves (2 g) and stirred for 89 h at 80° C., 24 h at 100° C., and 72 h at 110° C. After filtration, the reaction mixture was poured into a cold saturated aqueous solution of sodium bicarbonate (50 ml). The aqueous phase was extracted twice with ethyl acetate (50 ml) and the combined organic phases were washed with an saturated aqueous solution of sodium bicarbonate (50 ml) and with an aqueous NaCl solution (50 ml), dried (MgSO₄) and the solvent evaporated. FC (SiO₂, ethyl acetate) of the crude product (2.2 g) gave the desired bicyclic imidazole (0.27 g, 11% over three steps) and recovered azacyclododecan-2-one (0.44 g).

¹H-NMR (400 MHz, CDCl₃): [symbol] 7.00 (d, J=1.3), 6.80 (d, J=1.3), 3.91 (t, J=6.7, CH₂N), 2.68 (t, J=7.1, CH₂C═N), 1.93-1.81 (m, 4H), 1.46-1.24 (m, 12H).

¹³C-NMR (100 MHz, CDCl₃): [symbol] 148.92 (s), 127.74 (d), 117.80 (d), 43.19 (t), 29.14 (t), 26.72 (t), 25.10 (t), 24.68 (t), 23.93 (t), 23.21 (t), 22.95 (t), 22.87 (t), 22.74 (t).

MS (EI): 207 (7), 206 (45), 205 (19), 177 (25), 165 (41), 163 (72), 151 (25), 149 (56), 137 (56), 135 (46), 123 (97), 121 (67), 110 (44), 109 (69), 96 (100), 95 (48), 82 (77), 55 (44), 41 (56).

### EXAMPLE 3

### 6,7,8,9,10,11,12,13,14,15-decahydro-5H-imidazo[1,2-a][1]aza-cyclotridecine (Compound ID 64)

Prepared in three steps (10% yield) from laurinolactam following the general procedure as described in example 2.

¹H-NMR (400 MHz, CD₃OD): [symbol] 6.99 (d, J=1.3), 6.85 (d, J=1.3), 3.97 (t, J=6.9, CH₂N), 2.75 (t, J=7.3, CH₂C═N), 1.91-1.76 (m, 4H), 1.47-1.27 (m, 12H), 1.22-1.13 (m, 2H).

¹³C-NMR (100 MHz, CD₃OD): δ 147.81 (s), 125.40 (d), 119.29 (d), 44.14 (t), 27.56 (t), 25.32 (t), 25.23 (t), 25.10 (t), 25.06 (t, 2 C), 24.99 (t), 24.50 (t), 23.33 (t), 22.62 (t).

MS (EI): 221 (7), 220 (43), 219 (14), 205 (17), 179 (57), 177 (27), 165 (17), 163 (23), 151 (35), 149 (22), 137 (56), 135 (34), 123 (100), 121 (53), 110 (41), 109 (47), 96 (99), 95 (40), 82 (68), 55 (37), 41 (48).

IR: νₘₐₓ 3091, 2926, 2853, 1486, 1464, 1445, 1429, 1372, 1348, 1294, 1273, 1165, 1133, 1097, 1070, 980, 762, 735, 677 cm⁻¹.

UV (MeOH): λ (log ε) 210 (3.9), 281 (2.7).

### EXAMPLE 4

### 5,6,7,8,9,10,11,12-octahydro-4H-cycloundeca[d]oxazole (Compound ID 14) and 1,4,5,6,7,8,9,10,11,12-decahydrocycloundeca[d]imidazole (Compound ID 35)

At 20° C., a solution of cycloundecanone (4.76 g, 0.028 mol) in carbon tetrachloride (30 ml) was treated with a solution of bromine (4.5 g, 0.028 mol) in carbon tetrachloride (20 ml) and the resulting mixture was stirred for 1.5 h. The solvent was then evaporated giving the crude alpha-bromocycloundecanone (7.8 g).

At temperature below 50° C., an emulsion of crude alpha-bromocycloundecanone (7.8 g) in formamide (17 g) was treated dropwise with a solution of concentrated sulphuric acid (4.1 g) in formamide (17 g). After stirring for 2 h at 100° C., 3 h at 110° C., and 12 h at 20° C., the reaction mixture was poured into 2M aqueous sodium hydroxide (100 ml) and extracted twice with ethyl acetate (100 ml). The combined organic phases were washed twice with an aqueous solution of NaCl (100 ml), dried (MgSO₄) and the solvent evaporated. Ball-to-ball distillation (130° C., 0.08 mbar) of the crude product (5.9 g) followed by FC (SiO₂, hexane/methyl tent.-butyl ether 13:1 and hexane/ethyl acetate 20:1) gave the desired bicyclic oxazole (53 mg, 1%). FC (SiO₂, ethyl acetate/methanol 15:1) of the residue of the ball-ball distillation gave the desired bicyclic imidazole (42 mg, 1%).

### 5,6,7,8,9,10,11,12-octahydro-4H-cycloundeca[d]oxazole

¹H-NMR (400 MHz, CDCl₃): [symbol] 7.77 (s), 2.74-2.69 (m, 2H), 2.59-2.53 (m, 2H), 1.82-1.69 (m, 4H), 1.28-1.08 (m, 10H).

¹³C-NMR (100 MHz, CDCl₃): [symbol] 149.31 (d), 147.32 (s), 134.43 (d), 27.13 (t), 25.92 (t), 25.88 (t), 25.78 (t), 25.49 (t), 24.15 (t), 23.83 (t), 23.55 (t), 23.48 (t).

MS (EI): 193 (66), 178 (7), 176 (4), 164 (33), 150 (50), 148 (39), 136 (51), 122 (50), 109 (59), 97 (62), 96 (92), 95 (95), 83 (32), 81 (43), 67 (86), 55 (93), 41 (100).

### 1,4,5,6,7,8,9,10,11,12-decahydrocycloundeca[d]imidazole

¹H-NMR (400 MHz, CD₃OD): [symbol] 7.54 (s), 2.68-2.57 (m, 4H), 1.78-1.67 (m, 4H), 1.32-1.05 (m, 10H).

¹³C-NMR (100 MHz, CD₃OD): [symbol] 133.52 (d), 130.80 (s, 2 C), 26.94 (t), 26.73 (t, 2 C), 25.41 (t, 2 C), 23.32 (t, 2 C), 23.28 (t, 2 C).

MS (EI): 192 (42), 177 (8), 163 (20), 149 (48), 135 (47), 121 (45), 109 (31), 107 (40), 96 (54), 95 (100), 94 (58), 82 (33), 81 (32), 67 (17), 53 (20), 41 (32).

### EXAMPLE 5

### 5,6,7,8,9,10,11,12,13,14-decahydrocyclododeca[b]pyrazine (Compound ID 42)

Prepared as described in DE2117926 from cyclododecadione and ethylenediamine.

¹H-NMR (400 MHz, CDCl₃): [symbol] 8.33 (s, 2H), 2.88 (t, J=7.3, 4H), 1.92-1.84 (m, 4H), 1.57-1.46 (m, 4H), 1.44-1.33 (m, 8H).

¹³C-NMR (100 MHz, CDCl₃): [symbol] 156.17 (s), 141.43 (d), 31.10 (t), 27.56 (t), 25.59 (t), 24.86 (t), 22.90 (t).

MS (EI): 219 (16), 218 (100), 203 (3), 189 (3), 177 (7), 175 (15), 161 (27), 149 (41), 147 (28), 135 (72), 133 (37), 121 (37), 119 (27), 109 (29), 108 (66), 94 (7), 80 (11), 67 (12), 55 (16), 53 (15), 41 (39).

### EXAMPLE 6

### 5,6,7,8,9,10,11,12-octahydrocyclodeca[d]pyrimidine (Compound ID 26)

Prepared as described in DE1114497 starting from cyclodecanone via 1-chloro-2-formylcyclodecene.

¹H-NMR (400 MHz, CDCl₃): [symbol] 9.00 (s), 8.48 (s), 2.96 (t, J=7.6, 2H), 2.86 (t, J=7.6, 2H), 2.07-1.96 (m, 2H), 1.87-1.77 (m, 2H), 1.56-1.45 (m, 4H), 1.21-1.07 (m, 4H).

¹³C-NMR (100 MHz, CDCl₃): [symbol] 8168.72 (s), 157.55 (d), 156.16 (d), 133.41 (s), 31.28 (t), 28.72 (t), 27.63 (t), 26.62 (t), 26.10 (t), 25.49 (t), 20.92 (t), 20.28 (t).

MS (EI): 190 (10), 189 (10), 175 (14), 161 (24), 147 (100), 133 (51), 121 (28), 119 (22), 108 (75), 92 (7), 79 (10), 65 (12), 55 (6), 53 (14), 41 (20), 39 (24).

### EXAMPLE 7

### 5,6,7,8,9,10,11,12,13,14,15,16-dodecahydro-4H-cyclopentadeca[d]oxazole

Prepared as described in U.S. Pat. No. 3,956,196 starting from cyclopentadecanone.

¹H-NMR (400 MHz, CDCl₃): [symbol] 7.69 (s), 2.61 (t, J=7.3, 2H), 2.45 (t, J=7.5, 2H), 1.72-1.61 (m, 4H), 1.39-1.25 (m, 18H).

¹³C-NMR (100 MHz, CDCl₃): δ 148.61 (d), 147.20 (s), 134.15 (d), 27.65 (t), 27.29 (t), 27.20 (t), 26.97 (t, 2 C), 26.87 (t), 26.57 (t), 26.42 (t), 26.38 (t), 25.94 (t, 2 C), 24.98 (t), 23.97 (t).

MS (EI): 250 (5), 249 (26), 206 (13), 192 (8), 180 (5), 175 (3), 164 (8), 152 (20), 150 (13), 138 (31), 124 (19), 110 (26), 97 (100), 96 (38), 95 (24), 83 (10), 81 (17), 67 (31), 55 (50), 41 (54).

### EXAMPLE 8

### 4,5,6,7,8,9-hexahydrocycloocta[d]oxazole (Compound ID 60)

Prepared as described in DE2445387 (1973928, Plattier, M.; Shimizu, B.; Teisseire, P. J. Roure Bertrand Dupont) starting from cyclooctanone.

¹H-NMR (400 MHz, CDCl₃): [symbol] 7.66 (s), 2.84-2.79 (m, 2H), 2.75-2.70 (m, 2H), 1.84-1.74 (m, 4H), 1.59-1.47 (m, 4H).

¹³C-NMR (100 MHz, CDCl₃): [symbol] 147.98 (d), 147.18 (s), 133.63 (d), 26.26 (t), 25.81 (t), 25.67 (t), 25.46 (t, 2 C), 25.21 (t), 24.03 (t).

MS (EI): 152 (5), 151 (53), 150 (4), 123 (100), 122 (41), 109 (44), 108 (28), 96 (60), 95 (84), 82 (18), 80 (29), 67 (82), 55 (41), 41 (51).

### EXAMPLE 9

### Methyl 4-methyl-3-oxo-2-pentylcyclopentanecarboxylate (Compound ID 50)

A suspension of sodium hydride (60%, 22.6 g, 0.565 mol) in tetrahydrofuran (150 ml) was treated with dimethyl carbonate (40.75 g, 0.452 mol). The resulting mixture was brought to reflux, treated dropwise within 105 min. with 2-pentyl-2-cyclopenten-1-one (29 g, 0.181 mol), stirred for 2 h, cooled to 15° C., treated dropwise with 3M aqueous acetic acid (250 ml), acidified to pH 1 by addition of conc. HCl, and extracted three times with methyl tert-butyl ether (200 ml). The combined organic phases were washed with 2N aqueous sodium hydroxide, with a saturated aqueous solution of NaCl, dried (MgSO₄) and the solvent evaporated, giving the crude methyl 2-oxo-3-pentylcyclopent-3-enecarboxylate (37.6 g).

A solution of crude methyl 2-oxo-3-pentylcyclopent-3-enecarboxylate (37 g) in acetone (200 ml) was treated with potassium carbonate (69.1 g, 0.5 mol) and methyl iodide (53 g, 0.375 mol). The resulting mixture was stirred at reflux for 140 min., cooled and the solvent evaporated. The residue was added to 2N HCl and the mixture acidified to pH 1 by addition of conc. HCl and extracted three times with methyl tent-butyl ether (200 ml). The combined organic phases were washed with water, with a saturated aqueous solution of NaCl, dried (MgSO₄) and the solvent evaporated. Short-path Vigreux-distillation (125° C., 0.7 mbar) of the crude product (35 g) gave methyl 1-methyl-2-oxo-3-pentylcyclopent-3-enecarboxylate (16 g). Ball-to-ball distillation (200° C., 0.08 mbar) of the residue gave an additional fraction of methyl 1-methyl-2-oxo-3-pentylcyclopent-3-enecarboxylate (7.6 g).

A mixture of methyl 1-methyl-2-oxo-3-pentylcyclopent-3-enecarboxylate (19.3 g), acetone cyanohydrin (9.6 g, 0.113 mol) and sodium carbonate (0.68 g) in methanol (24 ml) and water (9.8 ml) was refluxed during 2 h, cooled, poured into ice/water, and extracted twice with methyl tert-butyl ether (100 ml). The combined organic phases were washed with water, with a saturated aqueous solution of NaCl, dried (MgSO₄) and the solvent evaporated giving the crude methyl 4-cyano-1-methyl-2-oxo-3-pentylcyclopentanecarboxylate (21.6 g).

A mixture of methyl 4-cyano-1-methyl-2-oxo-3-pentylcyclopentanecarboxylate (10.8 g), acetic acid (80 ml), conc. sulfuric acid (24 ml) and water (33 ml) was refluxed during 4.5 h, cooled, poured into ice/water. After addition of 2N aqueous NaOH solution, the mixture (pH 14) was extracted with methyl tert-butyl ether (100 ml). The aqueous phase was acidified with concentrated HCl to pH 1 and extracted three times with methyl tert-butyl ether (100 ml). The combined organic phases were washed with water, with a saturated aqueous solution of NaCl, dried (MgSO₄), the solvent evaporated, and the remaining acetic acid removed by ball-to-ball distillation of the residue (60° C., 0.1 mbar) giving the crude 4-methyl-3-oxo-2-pentylcyclopentanecarboxylic acid (8 g). At 20° C., a solution of crude 4-methyl-3-oxo-2-pentylcyclopentanecarboxylic acid (8 g) in dimethyl formamide was treated with potassium carbonate (12.3 g, 0.089 mol). The resulting suspension was stirred for 30 min., treated with methyl iodide, stirred for additional 2 h, poured into a saturated aqueous solution of NaCl, and extracted twice with methyl tent-butyl ether (100 ml). The combined organic phases were washed with water, with a saturated aqueous solution of NaCl, dried (MgSO₄), and the solvent evaporated. FC (SiO₂, hexane/MTBE 5:1) of the residue (8 g) gave methyl 4-methyl-3-oxo-2-pentylcyclopentanecarboxylate (4.1 g, 30% overall yield).

Boiling point: 90° C. (0.09 mbar).

¹³C-NMR (100 MHz, CDCl₃): [symbol] 219.15 (s), 175.11 (s), 52.01 (d), 45.14 (d), 43.98 (d), 33.96 (t), 31.70 (t), 29.41 (t), 26.13 (t), 22.38 (t), 14.08 (q), 13.94 (q).

MS (EI): 226 (2), 211 (1), 195 (2), 184 (2), 167 (18), 156 (19), 124 (3), 113 (7), 97 (100), 88 (6), 81 (8), 67 (8), 55 (19), 41 (13).

### EXAMPLE 10

### (E)-3-(cyclopropylmethylene)octan-2-one (Compound ID 2)

A solution of hexyl iodide (90 ml, 592 mmol) in triethyl phosphite (434 ml, 2.37 mol) was heated for 8 h at 150° C. The reaction mixture was then cooled to 20° C. and distilled using a Vigreux-distillation apparatus (11 mbar, bath temperature: 140-160° C.) giving diethyl hexylphosphonate (111.4 g, 85%). Boiling point: 126° C. (11 mbar).

¹³C-NMR (100 MHz, CDCl₃): δ 61.16 (t, J=6.6, 2 CH₂O), 31.14 (t, J=1.0, C (4)), 30.13 (t, J=16.6, C (3)), 25.57 (t, J=140.1, C (1)), 22.25 (t, C (5)), 22.23 (t, J=5.0, C (2)), 16.32 (q, J=5.8, 2 MeCH₂O), 13.83 (q, C (6)).

At −60° C., a solution of diisopropylamine (72.6 ml, 72%, 0.515 mol) in tetrahydrofuran (250 ml) was treated within 15 min. with a 1.6M solution of n-butyllithium in hexane (322 ml, 0.515 mol). The resulting solution was stirred 20 min. at −72° C. and treated with a solution of previously prepared diethyl hexylphosphonate (57.2 g, 0.257 mol) in tetrahydrofuran (150 ml). The resulting solution was stirred for 1 h at −72° C. and treated with a solution of ethyl acetate (37.8 ml, 0.386 mol) in tetrahydrofuran (100 ml). After stirring for 1 h at −70° C., the cooling bath was removed and the solution stirred for 1 h before being diluted with methyl t-butyl ether (250 ml) and acidified with aqueous 2M HCl (200 ml), aqueous 6M HCl (100 ml), and concentrated HCl to pH 6.4. The aqueous phase was extracted with methyl t-butyl ether (200 ml) and the combined organic phases were washed with aqueous NaCl solution (200 ml), dried (Na₂SO₄) and the solvent evaporated. Short-path Vigreux-distillation (0.07 mbar) of the crude product (74.3 g) gave diethyl 2-oxooctan-3-ylphosphonate (52.2 g, 77%). Boiling point: 107° C. (0.07 mbar).

¹³C-NMR (100 MHz, CDCl₃): δ 203.90 (s, J=4.1, CO), 62.56 (t, J=6.6, CH₂O), 62.47 (t, J=6.6, CH₂O), 53.75 (d, J=124.4, C (3)), 31.43 (t, C (6)), 31.08 (q, C (1)), 28.19 (t, J=14.9, C (5)), 26.39 (t, J=5.0, C (4)), 22.29 (t, C (7)), 16.34 (q, J=1.7, MeCH₂O), 16.33 (q, J=1.7, MeCH₂O), 13.91 (q, C (8)).

At 4° C., a mixture of a solution of NaOH (12.8 g, 0.32 mol) in water (27 ml) and dichloromethane (100 ml) was treated dropwise with a solution of diethyl 2-oxooctan-3-ylphosphonate (17.0 g, 64.3 mmol) and cyclopropanecarboxaldehyde (4.9 ml, 64.3 mmol) in dichloromethane (20 ml). The resulting mixture was stirred for 89 h at 20° C. and poured into ice/2M aqueous HCl (300 ml). The aqueous phase was extracted with cyclohexane (100 ml). The combined organic phases were washed twice with water (100 ml), dried (Na₂SO₄), and the solvent evaporated. FC (700 g SiO₂, hexane/methyl t-butyl ether 25:1) of the crude product (12.7 g) gave (E)-3-(cyclopropylmethylene)octan-2-one (6.25 g, 54%). Boiling point: 85° C. (0.08 mbar).

¹H-NMR (400 MHz, CDCl₃): δ 5.92 (d, J=10.4, H—C═C (3)), 2.38 (br. t, J=7.6, 2H—C (4)), 2.23 (s, C (1)H₃), 1.75-1.65 (m, H—CCH═), 1.43-1.25 (m, C (5)H₂, C (6)H₂, C (7)H₂), 1.01 (ddd, J=4.3, 6.6, 7.8, 2H), 0.88 (t, J=7.0, C (8)H₃), 0.63 (ddd, J=4.4, 6.5, 8.8, 2H).

¹³C-NMR (100 MHz, CDCl₃): δ 198.51 (s, C (2)), 148.99 (d, CH═C (3)), 140.51 (s, C (3)), 31.89 (t), 29.09 (t), 25.59 (t), 25.36 (q, C (1)), 22.54 (t), 14.01 (q, C (8)), 11.75 (d), 8.74 (t, 2 C).

MS (EI): 180 (1), 165 (19), 152 (27), 137 (6), 123 (12), 109 (24), 96 (40), 81 (25), 67 (17), 43 (100).

IR: νₘₐₓ 3007, 2956, 2928, 2859, 1659, 1632, 1457, 1392, 1357, 1262, 1174, 1123, 1049, 1022, 986, 954, 939, 847, 808, 722 cm⁻¹.

### EXAMPLE 11

### (E)-3-(cyclopropylmethylene)heptan-2-one (Compound ID 4)

Prepared as described in Example 10 in 38% yield from cyclopropanecarboxaldehyde and diethyl 2-oxoheptan-3-ylphosphonate (obtained from pentyl iodide and triethyl phosphite via diethyl pentylphosphonate). Boiling point: 50° C. (0.09 mbar).

¹H-NMR (400 MHz, CDCl₃): δ 5.92 (d, J=10.4, H—C═C (3)), 2.39 (br. t, J=7.5, 2H—C (4)), 2.24 (s, C (1)H₃), 1.75-1.65 (m, H—CCH═), 1.41-1.29 (m, C (5)H₂, C (6)H₂), 1.01 (ddd, J=4.3, 6.6, 7.8, 2H), 0.91 (t, J=7.3, C (7)H₃), 1.01 (dt, J=4.6, 6.6, 2H).

MS (EI): 166 (1), 151 (16), 138 (26), 123 (10), 109 (16), 96 (37), 95 (38), 81 (31), 67 (21), 53 (11), 43 (100).

### EXAMPLE 12

### (E)-3-(cyclopropylmethylene)nonan-2-one (Compound ID 6) and (1E,4E)-1-cyclopropyl-4-(cyclopropylmethylene)dec-1-en-3-one (Compound ID 56)

At 0° C., a mixture of a solution of NaOH (14.3 g, 0.36 mol) in water (22 ml) and dichloromethane (50 ml) was treated dropwise with a solution of diethyl 2-oxononan-3-ylphosphonate (obtained from heptyl iodide and triethyl phosphite via diethyl heptylphosphonate as described in Example 10, 19.9 g, 71 mmol) and cyclopropane-carboxaldehyde (4.9 ml, 64.3 mmol). The resulting mixture was stirred for 15 h at 20° C. and poured into ice/2M aqueous HCl. The aqueous phase was extracted three times with diethyl ether. The combined organic phases were washed with water, dried (Na₂SO₄), and the solvent evaporated. FC (700 g SiO₂, hexane/methyl t-butyl ether 25:1) of the crude product (14.1 g) gave (1E,4E)-1-cyclopropyl-4-(cyclopropylmethylene)dec-1-en-3-one (0.8 g, 5%) and (E)-3-(cyclopropylmethylene)nonan-2-one (2.3 g, 17%).

(E)-3-(cyclopropylmethylene)nonan-2-one (Boiling point: 87° C. at 0.08 mbar):

¹³C-NMR (100 MHz, CDCl₃): δ 198.55 (s, C (2)), 148.99 (d, CH═C (3)), 140.52 (s, C (3)), 31.72 (t), 29.39 (t, 2 C), 25.66 (t), 25.37 (q, C (1)), 22.62 (t), 14.05 (q, C (9)), 11.76 (d), 8.75 (t, 2 C).

MS (EI): 194 (1), 179 (12), 166 (17), 151 (5), 137 (5), 124 (6), 123 (16), 109 (35), 96 (60), 81 (29), 67 (21), 43 (100).

(1E,4E)-1-cyclopropyl-4-(cyclopropylmethylene)dec-1-en-3-one (Boiling point: 200° C. at 0.08 mbar):

¹³C-NMR (100 MHz, CDCl₃): δ 190.26 (s, C (3)), 151.84 (d), 147.27 (d), 140.58 (s, C (4)), 122.36 (d), 31.71 (t), 29.36 (t), 29.32 (t), 26.29 (t), 22.61 (t), 14.86 (d), 14.07 (q, C (10)), 11.78 (d), 8.74 (t, 2 C), 8.29 (t, 2 C).

MS (EI): 246 (2), 231 (3), 218 (5), 217 (5), 190 (13), 189 (13), 175 (10), 161 (22), 147 (56), 133 (71), 119 (15), 107 (27), 105 (32), 95 (47), 91 (46), 79 (44), 81 (30), 67 (100), 55 (49), 41 (95).

### EXAMPLE 13

### (E)-3-benzylideneheptan-2-one

As described in Example 10, the reaction of benzaldehyde and diethyl 2-oxoheptan-3-ylphosphonate (obtained from pentyl iodide and triethyl phosphite via diethyl pentylphosphonate) in 2:5 water/dichloromethane gave after FC, (E)-3-benzylideneheptan-2-one (22%) and (1E,4E)-4-benzylidene-1-phenyloct-1-en-3-one (19%). Boiling point: 90° C. (0.09 mbar).

¹H-NMR (400 MHz, CDCl₃): δ7.47 (s, H—C═C (3)), 7.45-7.31 (m, 5H), 2.53-2.47 (m, 2H—C (4)), 2.45 (s, C (1)H₃), 1.49-1.31 (m, 4H), 0.90 (t, J=7.2, C (7)H₃).

¹³C-NMR (100 MHz, CDCl₃): δ 200.26 (s, C (2)), 143.08 (s, C (3)), 139.34 (d, CH═C (3)), 135.84 (s), 129.20 (d, 2 C), 128.51 (d, 2 C), 128.47 (d), 31.32 (t), 26.15 (q, C (1)), 26.13 (t), 22.96 (t), 13.82 (q, C (7)).

MS (EI): 203 (6), 202 (41), 201 (35), 187 (20), 173 (5), 159 (35), 145 (16), 131 (16), 129 (53), 117 (72), 115 (57), 91 (52), 43 (100).

### EXAMPLE 14

### (E)-3-benzylideneoctan-2-one (compound ID 5) and (1E,4E)-4-benzylidene-1-phenylnon-1-en-3-one

As described in Example 10, the reaction of benzaldehyde and diethyl 2-oxooctan-3-ylphosphonate (obtained from hexyl iodide and triethyl phosphite via diethyl hexylphosphonate) in 1:2 water/dichloromethane gave after FC, (E)-3-benzylideneoctan-2-one (22%) and (1E,4E)-4-benzylidene-1-phenylnon-1-en-3-one (30%).

(E)-3-benzylideneoctan-2-one (Boiling point: 80° C. (0.08 mbar):

¹H-NMR (400 MHz, CDCl₃): δ 7.47 (s, H—C═C (3)), 7.44-7.32 (m, 5H), 2.51-2.46 (m, 2H—C (4)), 2.45 (s, C (1)H₃), 1.50-1.40 (m, 2H), 1.37-1.25 (m, 4H), 0.88 (t, J=7.1, C (8)H₃).

MS (EI): 217 (3), 216 (19), 201 (8), 173 (3), 159 (15), 145 (8), 129 (30), 117 (28), 115 (25), 91 (30), 43 (100).

(1E,4E)-4-benzylidene-1-phenylnon-1-en-3-one (Boiling point 180° C. at 0.07 mbar):

¹³C-NMR (100 MHz, CDCl₃): δ 193.15 (s, C (3)), 143.90 (s), 143.52 (d), 138.03 (d), 135.93 (s), 135.11 (s), 130.21 (d), 129.24 (d, 2 C), 128.90 (d, 2 C), 128.54 (d, 2 C), 128.41 (d), 128.26 (d, 2 C), 122.79 (d), 32.04 (t), 28.69 (t), 27.22 (t), 22.41 (t), 14.03 (q, C (9)).

### EXAMPLE 15

### (E)-3-benzylidenenonan-2-one (Compound ID 20)

Prepared as described in Example 10 in 16% yield from benzaldehyde and diethyl 2-oxononan-3-ylphosphonate (obtained from heptyl iodide and triethyl phosphite via diethyl heptylphosphonate). Boiling point: 108° C. (0.08 mbar).

¹³C-NMR (100 MHz, CDCl₃): δ 200.26 (s, C (2)), 143.09 (s, C (3)), 139.35 (d, CH═C (3)), 135.84 (s), 129.21 (d, 2 C), 128.52 (d, 2 C), 128.47 (d), 31.52 (t), 29.52 (t), 29.11 (t), 26.38 (t), 26.16 (q, C (1)), 22.58 (t), 14.05 (q, C (9)).

MS (EI): 231 (5), 230 (27), 229 (20), 215 (11), 187 (4), 173 (4), 159 (32), 145 (19), 129 (71), 117 (57), 115 (46), 91 (61), 43 (100).

### EXAMPLE 16

### 3-phenylmethylheptan-2-one

In an autoclave, a solution of (E)-3-benzylideneheptan-2-one (350 mg, 1.7 mmol, prepared as described in Example 13) in ethanol (5 ml) was stirred for 17 h under hydrogen (12 bars) in the presence of Pd/C (10%, 40 mg). The mixture was filtered over Celite and the solvent evaporated to give 3-phenylmethylheptan-2-one (350 mg, 99%).

Boiling point: 65° C. (0.11 mbar).

¹H-NMR (400 MHz, CDCl₃): δ 7.31-7.11 (m, 5H), 2.87 (dd, J=8.2, 12.6, 1H), 2.86-2.76 (m, 1H), 2.68 (dd, J=5.4, 12.8, 1H), 1.99 (s, C (1)H₃), 1.69-1.58 (m, 1H), 1.51-1.40 (m, 1H), 1.35-1.18 (m, 4H), 0.87 (t, J=6.9, C (7)H₃).

MS (EI): 204 (2), 189 (2), 148 (26), 147 (73), 131 (1), 129 (7), 117 (10), 115 (7), 105 (11), 91 (100), 65 (11), 43 (32).

IR: νₘₐₓ 3028, 3007, 2930, 2859, 1712, 1603, 1497, 1455, 1351, 1215, 1162, 1115, 1079, 1030, 946, 917, 741, 699 cm⁻¹.

### EXAMPLE 17

### 3-phenylmethyloctan-2-one (Compound ID 33)

Prepared in 75% yield as described in Example 16 by hydrogenation of (E)-3-benzylideneoctan-2-one (400 mg, 1.8 mmol, prepared as described in Example 14).

Boiling point: 70° C. (0.09 mbar).

¹H-NMR (400 MHz, CDCl₃): δ 7.30-7.11 (m, 5H), 2.87 (dd, J=8.3, 12.6, 1H), 2.85-2.77 (m, 1H), 2.68 (dd, J=5.4, 12.5, 1H), 1.99 (s, C (1)H₃), 1.68-1.57 (m, 1H), 1.50-1.39 (m, 1H), 1.33-1.19 (m, 6H), 0.87 (t, J=6.8, C (8)H₃).

MS (EI): 218 (2), 203 (2), 149 (3), 148 (34), 147 (86), 129 (7), 117 (11), 115 (7), 105 (12), 91 (100), 65 (10), 43 (35).

IR: νₘₐₓ 3064, 3028, 3007, 2929, 2858, 1712, 1603, 1496, 1455, 1352, 1162, 121, 1079, 1030, 950, 752, 700 cm⁻¹.

### EXAMPLE 18

### (E)-4-(2-acetylhept-1-enyl)benzonitrile (Compound ID 13)

Prepared as described in Example 10 in 10% yield from 4-cyanobenzaldehyde and diethyl 2-oxooctan-3-ylphosphonate (obtained from hexyl iodide and triethyl phosphite via diethyl hexylphosphonate). Boiling point: 205° C. (0.07 mbar).

¹³C-NMR (100 MHz, CDCl₃): δ 199.73 (s), 145.44 (s, C (2)), 140.55 (s), 136.56 (d, C (1)), 132.25 (d, 2 C), 129.56 (d, 2 C), 118.50 (s, CN), 111.84 (s), 31.93 (t), 28.81 (t), 26.49 (t), 26.26 (q, C (1)), 22.30 (t), 13.94 (q, C (7)).

MS (EI): 241 (14), 226 (13), 212 (8), 198 (8), 184 (21), 170 (23), 156 (31), 154 (34), 142 (53), 130 (12), 116 (30), 43 (100).

### EXAMPLE 19

### (E)-3-(naphthalen-2-ylmethylene)octan-2-one (Compound ID 67)

Prepared as described in Example 10 in 3% yield from 2-naphtaldehyde and diethyl 2-oxooctan-3-ylphosphonate (obtained from hexyl iodide and triethyl phosphite via diethyl hexylphosphonate). Boiling point: 220° C. (0.07 mbar).

¹³C-NMR (100 MHz, CDCl₃): δ 200.26 (s, C (2)), 143.31 (s, C (3)), 139.41 (d, CH═C (3)), 133.33 (s), 133.16 (s), 133.01 (s), 129.01 (d), 128.32 (d), 128.13 (d), 127.65 (d), 126.78 (d), 126.66 (d), 126.51 (d), 32.12 (t), 28.92 (t), 26.48 (t), 26.22 (q, C (1)), 22.40 (t), 14.05 (q, C (8)).

MS (EI): 267 (13), 266 (64), 265 (35), 251 (7), 223 (12), 209 (35), 195 (18), 179 (73), 167 (70), 165 (79), 152 (36), 141 (65), 128 (62), 115 (15), 43 (100).

### EXAMPLE 20

### (E)-3-(thiophen-2-ylmethylene)octan-2-one (Compound ID 38)

Prepared as described in Example 10 in 22% yield from 2-thiophencarboxaldehyde and diethyl 2-oxooctan-3-ylphosphonate (obtained from hexyl iodide and triethyl phosphite via diethyl hexylphosphonate). Boiling point: 115° C. (0.08 mbar).

¹H-NMR (400 MHz, CDCl₃): δ 7.62 (s, H—C═C (3)), 7.52 (dt, J=0.9, 5.2, 1H), 7.29 (ddd, J=0.6, 1.1, 3.6, 1H), 7.12 (dd, J=3.8, 7.1, 1H), 2.68-2.62 (m, 2H—C (4)), 2.43 (s, C (1)H₃), 1.50-1.31 (m, 6H), 0.91 (t, J=7.2, C (8)H₃).

MS (EI): 222 (20), 207 (7), 179 (16), 165 (13), 151 (9), 137 (14), 135 (12), 123 (42), 109 (15), 97 (31), 43 (100).

IR: νₘₐₓ 2955, 2927, 2859, 1657, 1609, 1456, 1420, 1389, 1356, 1259, 1204, 1124, 1053, 968, 943, 885, 857, 702, 634 cm⁻¹.

### EXAMPLE 21

### (E)-3-(2,2-dimethoxyethylidene)octan-2-one (Compound ID 9)

Prepared as described in Example 10 in 30% yield from dimethoxyacetaldehyde and diethyl 2-oxooctan-3-ylphosphonate (obtained from hexyl iodide and triethyl phosphite via diethyl hexylphosphonate). Boiling point: 60° C. (0.09 mbar).

¹H-NMR (400 MHz, CDCl₃): δ 6.41 (d, J=6.3, H—C═C (3)), 5.15 (d, J=6.3, H—C(OMe)₂), 3.37 (s, 2 MeO), 2.33 (s, C (1)H₃), 2.35-2.29 (m, 2H), 1.39-1.24 (m, 6H), 0.88 (t, J=6.9, C (8)H₃).

MS (EI): 214 (1), 183 (30), 171 (36), 157 (23), 139 (13), 125 (11), 111 (23), 95 (18), 75 (69), 55 (22), 43 (100).

IR: νₘₐₓ 2957, 2931, 2830, 1678, 1459, 1355, 1248, 1192, 1132, 1091, 1054, 963, 915, 723 cm⁻¹.

### EXAMPLE 22

### (E)-3-(2-(2-methyl-1,3-dioxolan-2-yl)ethylidene)octan-2-one (Compound ID 21)

Prepared as described in Example 10 in 24% yield from 2-(2-methyl-1,3-dioxolan-2-yl)acetaldehyde (prepared from ethyl acetoacetate by acetalisation with ethylene glycol in toluene in the presence of p-toluenesulfonic acid monohydrate followed by reduction using diisobutylaluminium hydride (1 M solution in hexane) in 10:1 hexane/tetrahydro-furan) and diethyl 2-oxooctan-3-ylphosphonate (obtained from hexyl iodide and triethyl phosphite via diethyl hexylphosphonate). Boiling point: 90° C. (0.09 mbar).

¹H-NMR (400 MHz, CDCl₃): δ 6.64 (t, J=7.2, H—C═C (3)), 4.03-3.96 (m, (OCH₂)₂), 2.61 (d, J=7.1, CH₂CH═), 2.32 (s, C (1)H₃), 2.30-2.24 (m, 2H), 1.36 (s, Me), 1.34-1.24 (m, 6H), 0.87 (t, J=6.9, C (8)H₃).

MS (EI): 225 (1), 87 (100), 53 (3), 43 (44).

IR: νₘₐₓ 2956, 2930, 2873, 1668, 1455, 1378, 1351, 1213, 1114, 1079, 1046, 948, 857, 784 cm⁻¹.

### EXAMPLE 23

### (E)-3-((tetrahydrofuran-3-yl)methylene)heptan-2-one (Compound ID 66)

Prepared as described in Example 10 in 30% yield from tetrahydro-3-furancarboxaldehyde and diethyl 2-oxoheptan-3-ylphosphonate (obtained from pentyl iodide and triethyl phosphite via diethyl pentylphosphonate). Boiling point: 75° C. (0.08 mbar).

¹H-NMR (400 MHz, CDCl₃): δ 6.46 (d, J=9.6, H—C═C (3)), 4.02-3.94 (m, 2H), 3.85 (dt, J=7.4, 8.1, 1H), 3.51 (dd, J=7.1, 8.6, 1H), 3.28-3.17 (m, 1H), 2.33-2.28 (m, 2H), 2.30 (s, C (1)H₃), 2.27-2.16 (m, 1H), 1.76 (dg, J=7.8, 12.4, 1H), 1.38-1.22 (m, 4H), 0.90 (t, J=6.8, C (7)H₃).

MS (EI): 196 (9), 181 (3), 165 (12), 151 (61), 138 (5), 125 (8), 123 (10), 109 (17), 95 (26), 81 (24), 67 (15), 55 (15), 43 (100).

IR: νₘₐₓ 2956, 2929, 2861, 1667, 1638, 1453, 1384, 1351, 1261, 1202, 1146, 1123, 1068, 956, 910, 723 cm⁻¹.

### EXAMPLE 24

### (Z)-1-(hex-3-enol)-1H-imidazole (Compound ID 23)

(Z)-3-hexenol (500 mg, 5 mmol) in 15 ml dry diethylether was treated with 1.69 ml of a 1:10 solution of PBr₃ in ether at −78° C. under Ar for 1 hour and at 0° C. for 5 h. The mixture was then poured into ice-water, extracted with hexane, washed with a saturated sodium bicarbonate solution and water. The crude (Z)-3-hexenyl bromide was mixed with imidazole (1.3 g, 19 mmol) in 10 ml dry THF containing a few mg of NaI and refluxed for 18 h. The solvent was evaporated under reduced pressure, the residue re-dissolved in methylene chloride, and the product extracted in 1N HCl/water. The water phase was adjusted to pH 9 with K₂CO₃, extracted with ethyl acetate and washed with water. The organic phase was evaporated under reduced pressure and the residue purified by FC(CH₂Cl₂/MeOH 93/7). (Z)-1-(hex-3-enyl)-1H-imidazole was obtained as a GC-pure oil (210 mg, 28%).

Rf 0.52 (CH₂Cl₂/MeOH 10/1). ¹H-NMR (400 MHz, CDCl₃): 7.50 (s, 1H); 7.04 (s, 1H); 6.91 (s, 1H); 5.50 (m, 1H); 5.28 (m, 1H); 3.95 (t, 2H); 2.49 (m, 2H); 1.92 (m, 2H); 0.89 (t, 3H). ¹³C-NMR (CDCl₃): 137.4; 135.9; 129.4; 123.8; 119.2; 47.37; 29.43; 20.90; 14.40.

GC-MS: 16.0 min, m/z 150.

### EXAMPLE 25

### (E)-1-(hex-3-enyl)-1H-imidazole

Following the same procedure as described in Example 24, starting from (E)-3-hexenol. The product was isolated as a GC-pure oil (156 mg, 20%).

Rf 0.47 (CH₂Cl₂/MeOH 10/1). ¹H-NMR (400 MHz, CDCl₃): 7.47 (s, 1H); 7.04 (s, 1H); 6.89 (s, 1H); 5.50 (m, 1H); 5.30 (m, 1H); 3.95 (t, 2H); 2.44 (m, 2H); 1.97 (m, 2H); 0.93 (t, J=7 Hz, 3H). ¹³C-NMR (CDCl₃): 137.39; 136.47; 129.48; 124.1; 119.23; 47.59; 34.71; 25.94; 13.97. GC-MS: 15.78 min, m/z 150.

### EXAMPLE 26

### 1-(hex-5-enyl)-1H-imidazole (Compound ID 41)

Following the general procedure describe in Example 24 starting from 1 bromohex-5-ene. The product was obtained as a GC-pure oil (647 mg 86%).

Rf 0.28 (CH₂Cl₂/MeOH 10/1). ¹H-NMR (400 MHz, CDCl₃): 7.43 (s, 1H); 7.02 (s, 1H); 6.87 (s, 1H); 5.72 (m, 1H); 4.95 (m, 2H); 3.90 (t, J=7 Hz, 2H); 2.05 (m, 2H); 1.76 (m, 2H); 1.36 (m, 2H). ¹³C-NMR (CDCl₃): 138.22; 137.41; 129.71; 119.16; 115.62; 47.27; 33.44; 30.80; 26.09. GC-MS: 16.13 min, m/z 150.

### EXAMPLE 27

### 2-(hept-6-enyl)pyrazine (Compound ID 57)

Methylpyrazine (940 mg, 912 10 mmol) was added to sodium amide (490 mg, 12.5 mmol) in 10 ml liquid NH₃ at −65° C. and the red mixture was stirred for 30 min. A solution of 1-bromohex-5-ene (7.5 mmol) in dry ether was added dropwise and the mixture was stirred for another hour. The reaction was quenched by addition of ammonium chloride (626 mg, 11.7 mmol) and NH₃ was evaporated by heating at ether reflux. The ether was removed and the residue extracted several times with ether. The combined ether phases were washed with water, dried over sodium sulfate, evaporated under vacuum and purified by FC (hexane/ethyl acetate 1/1). 2-(hept-6-enyl)pyrazine was isolated as a GC-pure oil (1.03 g, 78%).

Rf 0.52 (hexane/ethyl acetate 1/1). ¹H-NMR (400 MHz, CDCl₃): 8.48 (s, 1H); 8.45 (s, 1H); 8.39 (s, 1H); 5.78 (m, 1H); 5.00-4.94 (m, 2H); 2.81 (t, J=7 Hz, 2H); 2.04 (m, 2H); 1.75 (m, 2H); 1.41 (m, 4H). ¹³C-NMR (CDCl₃): 158.29; 144.96; 144.34; 142.46; 139.22; 114.82; 35.81; 33.99; 29.65; 29.12; 29.03. GC-MS: 16.23 min, m/z 176.

### EXAMPLE 28

### Evaluation of the test compounds as inhibitors of CYP2A13

Compounds that inhibit the activity of CYP2A13 are identified by using a standard reaction established for the enzyme. A known substrate is coumarin, and the product of the enzymatic reaction is 7-hydroxy-coumarin (Umbelliferone) which is strongly fluorescent. When a compound is added to the standard reaction and the formation of Umbelliferone is decreased, the compound is identified as an inhibitor, which can also be a competitive substrate of the enzyme. The compound is used at various concentrations and the concentration-dependent decrease in Umbelliferone formation allows to determine the concentration where the activity of the enzyme is reduced to the 50% level (IC50 value).

A test compound (details see Table 1) was incubated with CYP2A13 in the presence of a cytochrome P450 reductase. CYP2A13 and P450 reductase were employed in form of microsomes. CYP2A13 was produced in Sf9 cells using a recombinant baculovirus, under conditions known to the person skilled in the art, for example, as described in WO 2006/007751. P450 reductase is commercially available (BD Biosciences Gentest, USA). Preferably, the two enzymes are coexpressed in the same insect cells and microsomes prepared which contain both enzymes. The art of coexpression of two enzymes is known, and the coexpression CYP2A13 and P450 reductase is described in WO 2006/007751. Variability of activity was observed for high-titer recombinant virus batches, and optimal multiplicity of infection (MOI) has to be determined as known to the skilled person. An MOI of 4 for recombinant CYP2A13 baculovirus combined with an MOI of 3.5 for recombinant P450 reductase baculovirus routinely produced microsomes with considerable activity.

Microsomes were used which contained 7 pmoles CYP2A13. Tris buffer (1 M, pH 7.6) and water were added to give a buffer concentration of 0.1 M. The test compound was prepared as a 50 mM stock solution in acetonitrile. The concentration of the standard substrate coumarin was 0.006 mM. Several samples of the test compound were prepared at various concentrations to give different final concentrations in the reaction: 0, 0.005, 0.01, 0.02, 0.05, 0.1 and 0.2 mM. The mixture was incubated for 10 min at 37° C. prior to the initiation of the enzymatic reaction by the addition of 0.005 ml of a solution of 50 mM NADPH in water. The final total volume was 0.2 ml, which is suitable for microtiter plate measurements. The samples were incubated for 60 min at 37° C. After 60 min, the enzymatic reaction was stopped by the addition of 0.02 ml cold 50% trichloroacetic acid (TCA) and incubated at 4° C. for 15 min. 0.005 ml of a solution of 50 mM NADPH in water was added to the control reaction which corresponds to the reaction without test compound and without NADPH, and as a consequence, no Umbelliferone was formed. Denatured proteins and other insoluble parts were separated by centrifugation (10 min, 560×g, room-temperature).

The samples were analysed spectrofluorometrically which allows to detect the formation of Umbelliferone as the enzymatic product of coumarin at an excitation wavelength of 340 nm and an emission wavelength of 480 nm. A decrease of the fluorescent signal at 480 nm with respect to the control shows that the test compound is influencing enzymatic activity and confirms the nature of an inhibitor, since no metabolites have been detected. Graphical analysis of the data allows to calculate the concentration, where the test compound inhibits the enzyme to the level of 50% maximal activity (IC50 value).

TABLE 1
CYP2A13 inhibitor activity

| Comp. | |
|---|---|
| IC₅₀ | |
| (μM) | Chemical Structure |
| ID 1 0.3 μM | 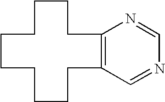 |
| ID 2 0.3 μM | 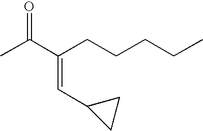 |
| ID 3 0.4 μM | 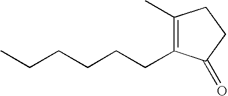 |
| ID 4 0.5 μM | 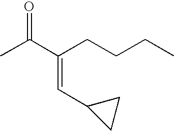 |
| ID 5 0.6 μM | 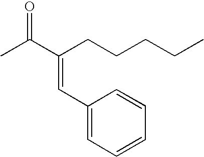 |
| ID 6 1.1 μM | 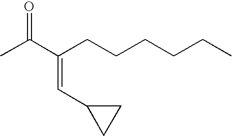 |
| ID 7 1.2 μM | 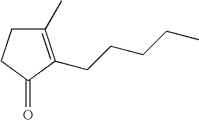 |
| ID 8 1.4 μM | 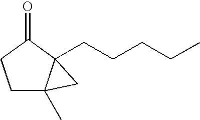 |
| ID 9 1.6 μM | 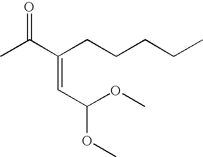 |
| ID 10 1.6 μM | 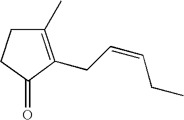 |
| ID 11 1.6 μM | 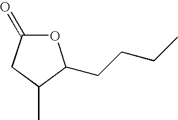 |
| ID 12 1.7 μM | 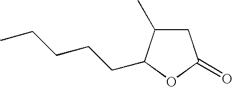 |
| ID 13 2.0 μM | 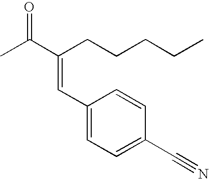 |
| ID 14 2.0 μM | 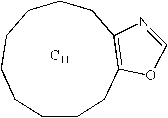 |
| ID 15 2.0 μM | 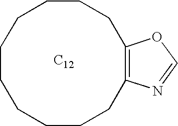 |
| ID 16 2.1 μM | 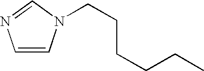 |
| ID 17 2.8 μM | 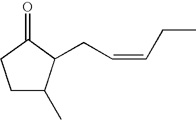 |
| ID 18 2.8 μM | 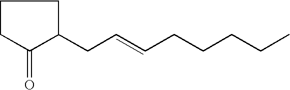 |
| ID 19 3.0 μM | 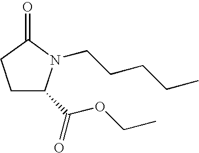 |
| ID 20 3.0 μM | 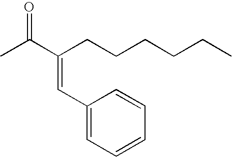 |
| ID 21 3.2 μM | 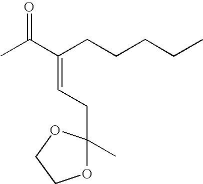 |
| ID 22 3.3 μM | 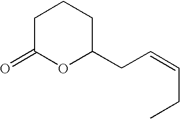 |
| ID 23 3.4 μM | 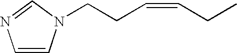 |
| ID 24 3.5 μM | 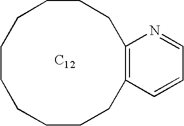 |
| ID 25 3.5 μM | 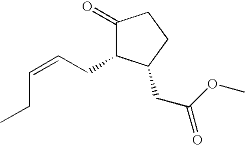 |
| ID 26 3.7 μM | 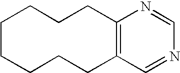 |
| ID 27 4.2 μM | 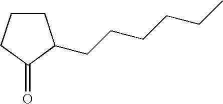 |
| ID 28 4.2 μM | 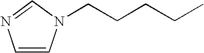 |
| ID 29 4.3 μM | 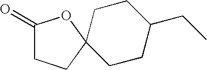 |
| ID 30 4.5 μM | 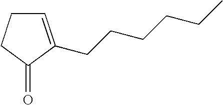 |
| ID 31 4.6 μM | 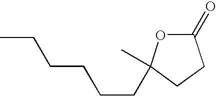 |
| ID 32 4.8 μM | 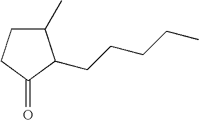 |
| ID 33 4.8 μM | 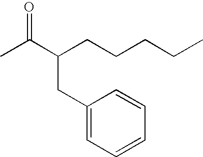 |
| ID 34 4.8 μM | 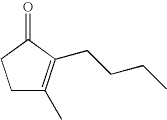 |
| ID 35 5.0 μM | 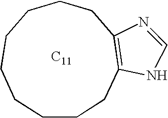 |
| ID 36 5.2 μM | 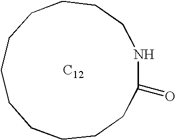 |
| ID 37 5.2 μM | 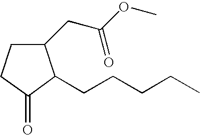 |
| ID 38 5.3 μM | 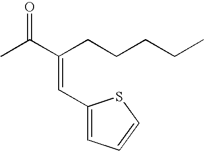 |
| ID 39 5.6 μM | 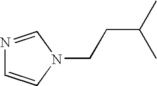 |
| ID 40 5.7 μM | 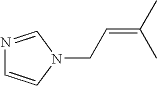 |
| ID 41 5.9 μM | 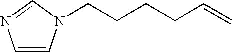 |
| ID 42 6.1 μM | 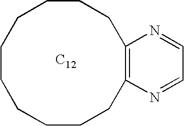 |
| ID 43 6.3 μM | 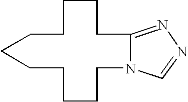 |
| ID 44 6.4 μM | 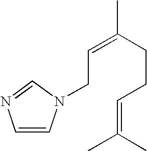 |
| ID 45 6.6 μM | 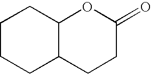 |
| ID 46 6.8 μM | 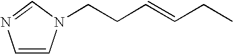 |
| ID 47 7.2 μM | 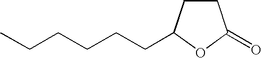 |
| ID 48 7.4 μM | 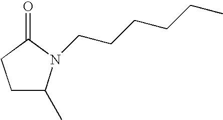 |
| ID 49 7.7 μM | 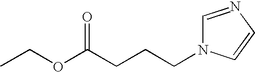 |
| ID 50 7.8 μM | 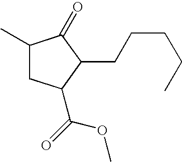 |
| ID 51 8.3 μM | 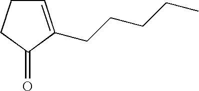 |
| ID 52 8.3 μM | 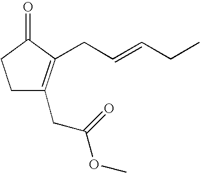 |
| ID 53 8.5 μM | 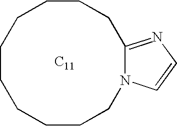 |
| ID 54 9.4 μM | 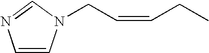 |
| ID 55 9.5 μM | 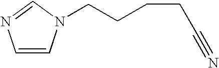 |
| ID 56 9.7 μM | 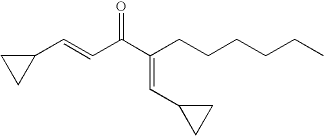 |
| ID 57 10.2 μM | 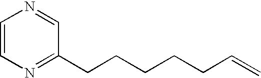 |
| ID 58 11.1 μM | 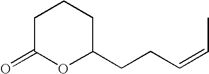 |
| ID 59 11.5 μM | 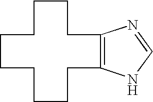 |
| ID 60 11.4 μM | 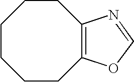 |
| ID 61 12.7 μM | 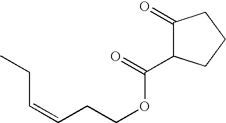 |
| ID 62 12.5 μM | 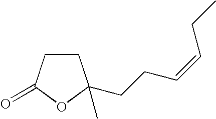 |
| ID 63 15.1 μM | 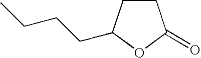 |
| ID 64 14.6 μM | 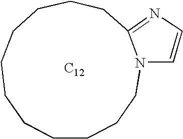 |
| ID 65 15.6 μM | 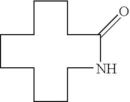 |
| ID 66 15.5 μM | 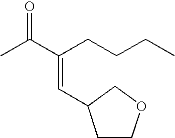 |
| ID 67 16.3 μM | 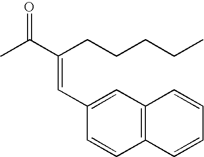 |
| ID 68 16.1 μM | 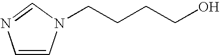 |
| ID 69 17.6 μM | 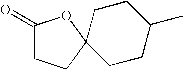 |

### EXAMPLE 29

### Evaluation of the test compounds as inhibitors of CYP2A6

Test compounds that inhibit the activity of CYP2A6 are identified by using the same principle as described in Example 28, first paragraph.

A test compound (details see list below) was incubated with CYP2A6 in the presence of a cytochrome P450 reductase. CYP2A6 and P450 reductase were employed in form of microsomes (BD Biosciences Gentest, USA). Microsomes were used which contained 2 pmoles CYP2A6 and an amount of NADPH-P450 reductase corresponding to cytochrome c reductase activity of 87 nmole/(min×mg protein). Tris buffer (Tris-(hydroxymethyl)aminomethane, 1 M, pH 7.6) and water were added to give a buffer concentration of 0.1 M. The test compound was prepared as a 50 mM stock solution in acetonitrile. The concentration of the standard substrate coumarin was 0.003 mM. Several samples of the test compound were prepared at various concentrations to give different final concentrations in the reaction: 0, 0.005, 0.01, 0.02, 0.05, 0.1 and 0.2 mM. (As obvious to the person skilled in the art, in cases where very good inhibitors were tested, lower concentrations were also used in order to have concentrations above and below the IC50 concentration present in the test wells.) The mixture was incubated for 10 min at 37° C. prior to the initiation of the enzymatic reaction by the addition of 0.005 ml of a solution of 50 mM NADPH in water. The final total volume was 0.2 ml, which is suitable for microtiter plate measurements. The samples were incubated for 60 min at 37° C. After 60 min, the enzymatic reaction was stopped by the addition of 0.02 ml cold 50% trichloroacetic acid (TCA) and incubated at 4° C. for 15 min. 0.005 ml of a solution of 50 mM NADPH in water was added to the control reaction which corresponds to the reaction without test compound and without NADPH, and as a consequence, no Umbelliferone was formed. Denatured proteins and other insoluble parts were separated by centrifugation (10 min, 560×g, room-temperature).

The samples were analysed spectrofluorometrically according to the procedure described in Example 28.

| | CYP2A6 inhibitor activity |
|---|---|
| Compound* | IC₅₀ |
| ID 1 | 9 μM |
| ID 2 | 7 μM |
| ID 3 | 13 μM |
| ID 4 | 3 μM |
| ID 5 | 141 μM |
| ID 6 | 8 μM |
| ID 10 | 54 μM |
| ID 11 | 56 μM |
| ID 15 | 53 μM |
| ID 23 | 7 μM |
| ID 28 | 2 μM |
| ID 33 | 148 μM |
| ID 44 | 6 μM |
| ID 54 | 7 μM |

*Chemical name and structure of the compounds are given in the description and Table 1.

### EXAMPLE 30

### Inhibition of NNK Metabolism

The catalytic activity of CYP2A13 in the presence or absence of an inhibitor according to the present invention was tested using radiolabeled [5-³H]NNK as the substrate according to the protocol described in Zhang et al. (2002) J. Pharmacol. Exp. Therap. 302: 416-423, also using NNK from Chemsyn Science Laboratories (Lenexa, Kans., USA).

Two metabolites, keto aldehyde (4-(3-pyridyl)-4-oxobutanal) and keto alcohol (4-hydroxy-1-((3-pyridyl)-1-butanone), which are formed from [5-³H]NNK by a CYP2A13-dependent α-carbon hydroxylation pathway can be detected by high-pressure liquid chromatography with an on-line radioactivity detector.

Procedure: Reaction mixtures contained 100 mM sodium phosphate, pH 7.4, 1 mM EDTA, an NADPH-generating system (5 mM glucose 6-phosphate, 3 mM MgCl₂, 1 mM NADPH, and 1.5 units of glucose-6-phosphate dehydrogenase), 10 μM NNK (containing 1 μCi [5-³H]NNK), 5 mM sodium bisulfite, and 10 μmol of purified, reconstituted CYP2A13 in a total volume of 0.4 ml. CYP2A13 was reconstituted with rat NADPH-P450 reductase, at a ratio of 1:4 (P450/reductase). Each test compound, i.e. Compound ID 1, 2, and 3, was diluted to 50 mM in acetonitrile based on molecular weight and further diluted to 400 μM by adding 1.2 μl to 148.8 μl water. This concentration was used to reach the final reaction concentrations (10 μl was added for 10 μM and 1 μl was added for 1 μM). The final concentration of acetonitrile was 0.02% in the 10 μM reactions and 0.002% in the 1 μM reactions. Reactions were carried out for 10 minutes at 37° C. before being terminated with 50 μl each saturated barium hydroxide and 25% zinc sulfate. The results are shown in Table 2 below.

TABLE 2
Blocking of the metabolic activation of NNK

| | controls | |
|---|---|---|
| | Keto aldeyde (pmol/ | Keto alcohol (pmol/ |
| Inhibitor | min/pmol CYP) | min/pmol CYP) |
| No inhibitor | 3.46 | 1.33 |
| 1 μM Compound ID1, 2, 3 | N.D. | N.D. |
| 0.002% acetonitrile | 3.28 | 1.06 |
| 10 μM Compound ID1, 2, 3 | N.D. | N.D. |
| 0.02% acetonitrile | 3.05 | 0.95 |

(N.D. = none detected)

Compound ID 1=5,6,7,8,9,10,11,12,13,14-decahydrocyclododeca[d]pyrimidine
Compound ID 2=(E)-3-(cyclopropylmethylene)octan-2-one
Compound ID 3=2-hexyl-3-methylcyclopent-2-enone

The inhibition results clearly demonstrate that inhibitors, i.e. compounds of formula (I) are efficient inhibitors of CYP2A13 with an IC50 value clearly below 1 μM for NNK as substrate, since at 1 μM the enzyme was completely inhibited. Acetonitrile which was used as a solvent slightly affects the activity of CYP2A13 at the concentrations used in the enzymatic assay.

### EXAMPLE 31

### Inhibition of Human CYP2B6

Test compounds that inhibit the activity of CYP2B6 are identified by using the same principle as described in Example 28, first paragraph.

A test compound (details see Table 3) was incubated with CYP2B6 in the presence of a cytochrome P450 reductase. CYP2B6 and P450 reductase are produced using recombinant baculoviruses and co-expressing the two proteins in Sf9 insect cells as described in Example 28. Alternatively, microsomes containing CYP2B6 and the reductase are commercially available (BD Biosciences Gentest, USA). Microsomes were used which contained 1.5 pmoles CYP2B6. Potassium phosphate buffer final concentration was 100 mM, (1M stock, pH 7.4). The test compound was prepared as a 50 mM stock solution in acetonitrile. The concentration of the standard substrate 7-ethoxy-4-trifluoromethyl-coumarin was 6 μM. Several samples of the test compound were prepared at various concentrations to give different final concentrations in the reaction: 0, 0.005, 0.01, 0.02, 0.05, 0.1 and 0.2 mM. (As obvious to the person skilled in the art, in cases where very good inhibitors were tested, lower concentrations were also used in order to have concentrations above and below the IC50 concentration present in the test wells.) The mixture was incubated for 10 min at 37° C. prior to the initiation of the enzymatic reaction by the addition of 0.005 ml of a solution of 50 mM NADPH in water. The final total volume was 0.2 ml, which is suitable for microtiter plate measurements. The samples were incubated for 40 min at 37° C. After 40 min, the enzymatic reaction was stopped by the addition of 75 μl of 0.5M Tris-base/acetonitrile (18:72). 0.005 ml of a solution of 50 mM NADPH in water was added to the control reaction which corresponds to the reaction with test compound and enzyme but without NADPH, and as a consequence, no 4-trifluoromethyl-umbelliferone was formed. Denatured proteins and other insoluble parts were separated by centrifugation (5 min, 1800 rpm, at 10° C.).

The samples were analysed spectrofluorometrically which allows to detect the formation of 4-trifluoromethyl-umbelliferone as the enzymatic product at an excitation wavelength of 410 nm and an emission wavelength of 510 nm. A decrease of the fluorescent signal at 510 nm with respect to the control shows that the test compound is influencing enzymatic activity and confirms the nature of an inhibitor, which can also be an alternative substrate. Graphical analysis of the data allows to calculate the concentration, where the test compound inhibits the enzyme to the level of 50% maximal activity (IC50 value). The results are shown in Table 3 below.

TABLE 3
CYP2B6 inhibitor activity

| Comp. | |
|---|---|
| IC₅₀ | |
| (μM) | Chemical Structure |
| ID 1 2.7 μM | 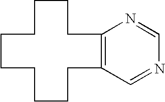 |
| ID 2 4.0 μM | 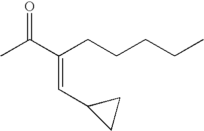 |
| ID 4 3.9 μM | 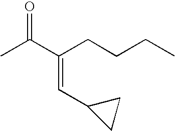 |
| ID 6 3.5 μM | 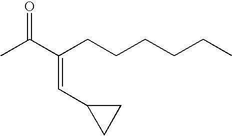 |
| ID 15 5.4 μM | 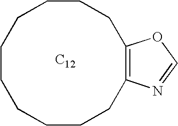 |
| ID 16 0.6 μM | 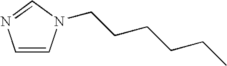 |
| ID 23 2.2 μM | 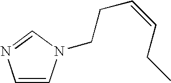 |
| ID 41 1.9 μM | 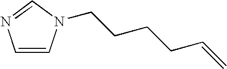 |
| ID 44 0.1 μM | 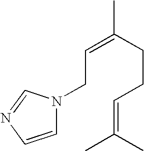 |
| ID 57 0.6 μM | 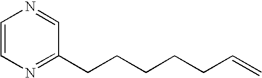 |

1. A tobacco product comprising a compound of formula (I) 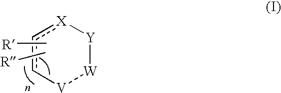
wherein
n is 0 or an integer from 1 to 12;
the dashed lines representing independently a bond or no bond;
R′ is H, C₁-C₁₀ alkyl, C₂-C₁₀ alkenyl, —CH₂—C(O)—(C₁-C₁₀)alkyl, or —(CH₂)ₖ—COO—(C₁-C₁₀)alkyl, wherein k is 0 or 1; and
R″ is H, C₁-C₁₀ alkyl; or
R′ and R″ together represent a bivalent group —(CH₂)ₐ— wherein “a” is 1-5, forming together with the carbon atom(s) to which they are attached cycloalkyl optionally substituted with C₁-C₃ alkyl, or C₁-C₃ alkoxy;
I) —X—Y— represents a bivalent group selected from 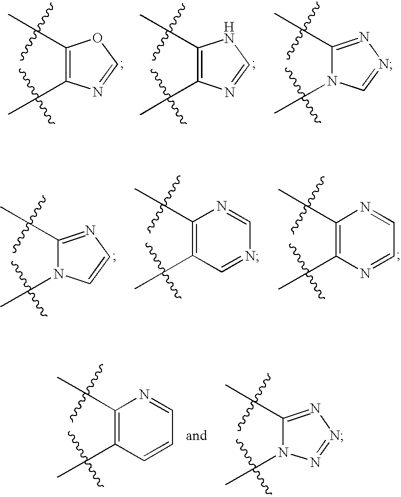
II) Y is carbonyl and
X is O, NH, CHR², CR² wherein R² is H, C₁-C₁₀ alkyl, C₂-C₁₀ alkenyl, C₂-C₁₀ alkynyl, or —COO—R¹¹ wherein R¹¹ is C₁-C₁₀ alkyl or C₂-C₁₀ alkenyl, or X is NR³, wherein R³ is C₁-C₁₀ alkyl or C₂-C₁₀ alkenyl;
or
III) Y is —CR¹═, wherein R¹ is H, C₁-C₁₀ alkyl, C₂-C₁₀ alkenyl, or cycloalkylalkyl; and
X is O, N, or NR^{S}, wherein R⁵ is H, C₁-C₁₀ hydroxyalkyl, C₁-C₁₀ cyanoalkyl, C₁-C₁₀ alkyl, C₂-C₁₀ alkenyl, C₂-C₁₀ alkynyl, —(CH₂)ₘ—COO—R¹², wherein m is 1, 2, 3, 4, or 5 and R¹² is H, or C₁-C₁₀ alkyl;
and
i) if the dashed line V [symbol] W represents a bond then
V is selected from O, N, —CH₂—, —CR⁴═ wherein R⁴ is H, or C₁-C₃ alkyl, —CR⁶R⁷— wherein R⁶ is H, or C₁-C₆ alkyl, and R⁷ is H, or R⁷ and R′ together represent a bivalent group selected from —O— and —CH₂— forming a 3-membered ring; and
W represents a direct bond from Y to V, or is —CH₂—, —CHR″— or —CH═;
ii) if the dashed line V [symbol] W is no bond;
W is C₁-C₃ alkyl, C₂-C₇ alkenyl, cycloalkylvinyl comprising from 5 to 7 carbon atoms, arylvinyl comprising from 5 to 7 carbon atoms, phenyl, C₁-C₃ alkoxy, or C₂-C₃ alkenyloxy; and
A) V is —CR⁸R⁹R¹⁰ wherein R⁸, R⁹, R¹⁰ are hydrogen, R⁸ and R⁹ are methyl and R¹⁰ is hydrogen or methyl; or R⁸ and R⁹ representing independently H, or C₁-C₆ alkoxy and R¹⁰ is C₁-C₆ alkoxy;
B) V is a 3-6 membered monocyclic or 6-10 membered bicyclic hydrocarbon ring wherein up to two, i.e. 0, 1 or 2, C atom(s) are replaced by a hetero atom selected from S, O, and N;
C) V is a 3-6 membered monocyclic or a 6-10 membered bicyclic hydrocarbon ring wherein up to two, i.e. 0, 1 or 2, C atom(s) are replaced by a hetero atom selected from S, O, and N, and the ring is substituted with one or two groups selected from CN, halogen, C₁-C₃ alkoxy, C₁-C₃ alkyl and —COOR, wherein R is hydrogen, methyl, ethyl, propyl or isopropyl;
D) V is a bivalent residue —CH₂—CH₂— forming together with the carbon atom of X which is in alpha-position to the carbonyl group (Y) a cyclobutan or cyclopentan ring; or
E) V is —C(O)R¹³ wherein R¹³ is C₁-C₃ alkyl, or C₁-C₃ alkoxy.

2. A product according to claim 1 wherein the compound of formula (I) is selected from the group of compounds wherein
R′ and R″ are hydrogen,
n is an integer from 3 to 11;
the dashed line V [symbol] W represents a bond wherein W and V are —CH₂—; and
Y is carbonyl and X is NH; or
—X—Y— represents a bivalent group selected from

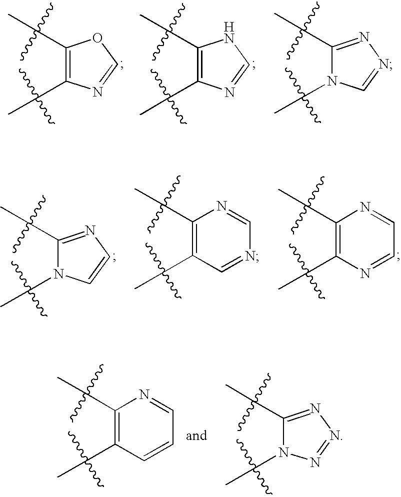

3. A product according to claim 1 wherein the compound of formula (I) is selected from the group of compounds wherein
n is 0 or 1;
the dashed line V [symbol] W represents a bond;
W represents a direct bond from Y to V, or is —CH₂—, —CHR″— or —CH═;
V is selected from O, N, —CH₂—, —CR⁴═ wherein R⁴ is H, or C₁-C₃ alkyl, CR⁶R⁷ wherein R⁶ is H, or C₁-C₆ alkyl, and R⁷ is H, or R⁷ and R′ together represent a bivalent group selected from —O— and —CH₂— forming a 3-membered ring;
R′ is H, C₁-C₁₀ alkyl, C₂-C₁₀ alkenyl, —CH₂—C(O)—(C₁-C₁₀)alkyl, or —(CH₂)ₖ—COO—(C₁-C₁₀)alkyl, wherein k is 0 or 1; and
R″ is H, C₁-C₁₀ alkyl; or
R′ and R″ together represent a bivalent group —(CH₂)ₐ— wherein a is 1-5, forming together with the carbon atom(s) to which they are attached a cycloalkyl optionally substituted with C₁-C₃ alkyl, or C₁-C₃ alkoxy; and
I) Y is carbonyl and
X is O, NH, CHR², CR² wherein R² is H, C₁-C₁₀ alkyl, C₂-C₁₀ alkenyl, C₂-C₁₀ alkynyl, or —COO—R¹¹ wherein R¹¹ is C₁-C₁₀ alkyl or C₂-C₁₀ alkenyl, or X is NR³, wherein R³ is C₁-C₁₀ alkyl or C₂-C₁₀ alkenyl; or
II) Y is —CR¹═, wherein R¹ is H, C₁-C₁₀ alkyl, C₂-C₁₀ alkenyl, or cycloalkylalkyl; and
X is O, N, or NR⁵, wherein R⁵ is H, C₁-C₁₀ hydroxyalkyl, C₁-C₁₀ cyanoalkyl, C₁-C₁₀ alkyl, C₂-C₁₀ alkenyl, C₂-C₁₀ alkynyl, —(CH₂)ₘ—COO—R¹², wherein m is 1, 2, 3, 4, or 5 and R¹² is H, or C₁-C₁₀ alkyl.

4. A product according to claim 1 wherein the compound of formula (I) is selected from the group of compounds wherein
Y is carbonyl;
R′ is H, C₁-C₁₀ alkyl, or C₂-C₁₀ alkenyl; and R″ is H, C₁-C₁₀ alkyl; or
R′ and R″ together represent a bivalent group —(CH₂)ₐ— wherein a is 1-5, forming together with the carbon atom(s) to which they are attached cycloalkyl optionally substituted with C₁-C₃ alkyl, or C₁-C₃ alkoxy; and
i) X is oxygen; the dashed line V [symbol] W represents a bond wherein W represents a direct bond from Y to V and V is —CH₂—; n is 1 or 2, or
ii) X is CHR² wherein R² is hydrogen; the dashed line V [symbol] W represents a bond wherein W represents a direct bond from Y to V and V is oxygen; n is 1 or 2; or
iii) X is oxygen; the dashed line V [symbol] W represents a bond; W and V are —CH₂—; n is 0 or 1; or
iv) X is CHR² wherein R² is hydrogen; the dashed line V [symbol] W represents a bond wherein W represents a direct bond from Y to V and V is oxygen; n is 0 or 1.

5. A product according to claim 1 wherein the compound of formula (I) is selected from the group of compounds wherein
R′ and R″ are hydrogen;
n is 1;
the dashed line V [symbol] W represents a bond wherein W represents a direct bond from Y to V and V is N, Y is —CR¹═ wherein R¹ is hydrogen; and
X is NR^{S}, wherein R⁵ is C₁-C₁₀ hydroxyalkyl, C₁-C₁₀ cyanoalkyl, C₁-C₁₀ alkyl, C₂-C₁₀ alkenyl, C₂-C₁₀ alkynyl, —(CH₂)ₘ—COO—R¹², wherein m is 1, 2, 3, 4, or 5 and
R¹² is H, C₁-C₁₀ alkyl.

6. A product according to claim 1 wherein the compound of formula (I) is selected from the group of compounds wherein
n is 0 or 1;
the dashed lines represent independently a bond or no bond with the proviso that the dashed line V [symbol] W is no bond;
R′ is H; and R″ is H, or C₁-C₄ alkyl; or
R′ and R″ together represent a bivalent group —(CH₂)ₐ— wherein “a” is 1-5, forming together with the carbon atoms to which they are attached a cycloalkyl;
Y is carbonyl;
X is CHR² or CR² wherein R² is H, C₁-C₁₀ alkyl;
W is C₁-C₃ alkyl, C₂-C₇ alkenyl, cycloalkylvinyl comprising from 5 to 7 carbon atoms, arylvinyl comprising from 5 to 7 carbon atoms, phenyl, C₁-C₃ alkoxy, or C₂-C₃ alkenyloxy; and
A) V is CR⁸R⁹R¹⁰ wherein R⁸, R⁹, R¹⁰ are hydrogen, R⁸ and R⁹ are methyl and R¹⁰ is hydrogen or methyl; or R⁸ and R⁹ representing independently H, or C₁-C₆ alkoxy and R¹⁰ is C₁-C₆ alkoxy;
B) V is a 3-6 membered monocyclic or 6-10 membered bicyclic hydrocarbon ring wherein up to two C atom(s) are replaced by a hetero atom selected from S, O, and N;
C) V is a 3-6 membered monocyclic or 6-10 membered bicyclic hydrocarbon ring wherein up to two C atom(s) are replaced by a hetero atom selected from S, O, and N, and the ring is substituted with one or two groups selected from CN, halogen, C₁-C₃ alkoxy, C₁-C₃ alkyl and —COOR, wherein R is hydrogen, methyl, ethyl, propyl or is isopropyl;
D) V is a bivalent residue —CH₂—CH₂— forming together with the carbon atom of X which is in alpha-position to the carbonyl group (Y) a cyclobutan or cyclopentan ring; or
E) V is —C(O)R¹³ wherein R¹³ is C₁-C₃ alkyl, or C₁-C₃ alkoxy.

7. A product according to claim 1 wherein the compound of formula (I) is selected from the group of compounds wherein
Y is carbonyl;
dashed line V [symbol] W is no bond, W is CH₃ or cyclopropylethenyl, X is CR² or CHR² wherein R² is C₃-C₁₀ alkyl;
n is 0 or 1, and
V is cyclopropyl, phenyl, naphthyl, furanyl, thienyl, tetrahydrofuranyl, 2-methyl dioxolan-2-yl, or
phenyl substituted with one or two groups selected from CN, halogen, C₁-C₃ alkoxy, C₁-C₃ alkyl and —COOR, wherein R is hydrogen, methyl, ethyl, propyl or is isopropyl, or
V is CR⁸R⁹R¹⁰ wherein R⁸ is hydrogen and R⁹ and R¹⁰ representing independently C₁-C₆ alkoxy.

8. A method comprising the step of disseminating a compound of formula (I) according to claim 1 into a room in the presence of tobacco smoke.

9. A pharmaceutical composition comprising a compound of formula (I) according to claim 1, and a pharmaceutically acceptable carrier.

10. A method of preparing a pharmaceutical composition comprising the step of; including a compound of formula (I) into the pharmaceutical composition.
